(19) United States
(12) Patent Application Publication
(10) Pub. No.: US 2018/0003572 A1
(43) Pub. Date: Jan. 4, 2018

(54) Sensor-Based Systems and Methods for Monitoring Temperature Sensitive Products

(71) Applicant: Exotag Inc., Del Mar, CA (US)

(72) Inventors: Emmanuel Garsd, Oceanside, CA (US); Zachary Gemmell, Del Mar, CA (US); Barrett Larson, Palo Alto, CA (US); Sergii Lapin, San Diego, CA (US); Konstantin Dubovenko, La Mesa, CA (US); Jeffrey Kuna, San Francisco, CA (US)

(73) Assignee: Exotag Inc., Del Mar, CA (US)

(21) Appl. No.: 15/640,189

(22) Filed: Jun. 30, 2017

Related U.S. Application Data
(60) Provisional application No. 62/357,833, filed on Jul. 1, 2016.
(60) Provisional application No. 62/435,322, filed on Dec. 16, 2016.

(51) Int. Cl. G01K 13/00; G01N 33/49

(52) U.S. Cl. CPC G01K 13/00; G01N 33/49

(57) ABSTRACT

A temperature measurement device is provided for monitoring the temperature of a temperature-sensitive product, e.g., a blood product. The temperature measurement device may include multiple different types of temperature sensors, e.g., at least two of the following types of temperature sensors: (a) a product-interfacing temperature sensor in thermal contact with the product, (b) an on-chip temperature sensor of a microprocessor or microcontroller, and/or (c) an ambient temperature sensor configured to measure an ambient temperature external to the product. The temperature measurement device may further include a processor configured to execute computer instructions to receive sensor signals from the multiple types of temperature sensors on the device, determine a product temperature of the product based at least on signals from the multiple temperature sensors, compare the determined product temperature with one or more threshold values, and determine whether to generate a notification based on the results of the comparison.

### RELATED APPLICATIONS

This application claims priority to (a) U.S. Provisional Patent Application Ser. No. 62/357,833 filed Jul. 1, 2016 and (b) U.S. Provisional Patent Application Ser. No. 62/435,322 filed Dec. 16, 2016, the entire contents of which applications are hereby incorporated by reference for all purposes.

### TECHNICAL FIELD

The present disclosure relates, in general, to the field of sensor-based monitoring systems for monitoring product characteristics, for example, sensor-based systems and methods for monitoring the temperature of a temperature sensitive product, e.g., a blood product.

### BACKGROUND

Temperature-sensitive products include substances or items that are sensitive to temperature changes or reaching high or low temperatures that may be detrimental to the qualities or usefulness of such products. Blood products, e.g., blood stored in blood bags or packs, are one example of a temperature-sensitive product. This document largely focus on the example of blood products, but it should be understood that any of the discussions and teachings herein may be similarly applied to various other types of temperature-sensitive products, such as for example: blood contained in a bag or other packaging), vaccines, pharmacologic agents, proteins, spinal fluid, bile, urine, breast milk, any other biofluids, chemicals, food, perishable items, or any other suitable liquids, gasses, or solids.

Temperature-sensitive products may be monitored, e.g., to manage the temperature within a desired temperature range and/or to identify or prevent the occurrence of undesirable temperature conditions. Conventional techniques for monitoring temperature-sensitive products include, for example, chemical indicators, handheld thermometers, “smart” coolers, or by following simple “rules of thumb.” Some example techniques and common drawbacks are discussed below.

Chemical Indicators:

Chemical indicators are typically designed to identify temperature threshold violations that may require disposal of a blood product. One example chemical indicator currently on the market is the Safe-T-Vue® product by William Laboratories (http://williamlabs.com/products/safe-t-vue/). Chemical indicators may suffer from one or more of the following drawbacks. First, chemical indicators may have a propensity for false positives. Second, the linear nature of measurements using chemical indicator typically cannot adjust to complex product degradation. Third, chemical indicators may provide ambiguous indications between two different states. For example, a chemical indicator may display an ambiguous color between two colors that indicate two different states. Fourth, chemical indicators typically measure only the surface of the product, which is often not representative of the core temperature of the product and thus can lead to false positives or negatives.

Handheld Temperature Measurement Devices

Commonly used handheld devices for measuring the temperature of temperature-sensitive products include handheld thermometers and infrared (IR) guns. The usual practice for measuring the temperature of a blood product is to simply measure the surface temperature of the product directly (without physical contact with the product) with a handheld thermometer or IR gun when the product leaves from or arrives at a storage facility, e.g., a blood bank at a hospital. Handheld temperature measurement device may suffer from one or more of the following drawbacks. First, like chemical indicators, handheld temperature measurement devices may have a propensity for false positives. Second, handheld temperature measurement devices typically provide a direct measurement of a specific point of the temperature-sensitive product, which does not account for temperature variance across the entire surface or volume of the product. Third, handheld measurement devices typically provide limited measurement data points (e.g., a single measurement upon arrival at or departure from a storage facility), which may fail to provide a useful thermal history of the product, including the extent of exposure to certain temperatures. Fourth, like chemical indicators, handheld measurement devices typically provide a surface temperature measurement, which is often not representative of the core temperature of the product and thus can lead to false positives or negatives.

Smart Cooler

A cooler with built in electronics for cold chain product tracking, referred to as a “smart cooler,” may be used to monitor certain temperature-sensitive products while they reside inside or within the vicinity of the cooler. Such smart coolers for cold-chain monitoring may suffer from one or more of the following drawbacks. First, the temperature of the product cannot be directly tracked when the product is outside of the cooler, often requiring the cooler to make assumptions about the temperature and handling conditions that the product is exposed to. Second, smart cooler systems are typically expensive, requiring significant investment. Third, a smart cooler system may have specific maintenance requirements that may require infrastructure changes and expensive employee training.

30-Minute Rule

A practice referred to as the “30-minute rule,” is sometimes used to estimate the time of exposure of a product to warm ambient temperature, and to label the product as “waste” when a 30 minute exposure threshold is reached. Relying on the 30-minute rule has various drawbacks. First, the rule is not based on empirical data for making a reliable conclusion about the actual state of a particular product. Second, the rule imposes stringent requirements that may complicate the relevant workflow and may be detrimental especially in hectic situations, such as many medical situations. Third, the rule generally creates a high probability of false positives and false negatives and promote unnecessary wastage of products.

### SUMMARY

The present application discloses sensor-based temperature monitoring devices, systems, and methods for monitoring temperature sensitive substances or materials, such as blood products (e.g., blood bags/packs), for example. The substances or materials monitored by the disclosed devices, systems, and methods are referred to herein as “Products.” A Product may include blood (e.g., blood contained in a bag or other packaging), vaccines, pharmacologic agents, proteins, spinal fluid, bile, urine, breast milk, any other biofluids, chemicals, food, perishable items, or any other suitable liquids, gasses, or solids.

The disclosed temperature monitoring devices, systems, and methods may address any one or multiple drawbacks of conventional temperature monitoring devices, systems, and methods, e.g., such as any of the various drawbacks of conventional techniques discussed above in the Background section. Some embodiments of the temperature monitoring devices, systems, and methods disclosed herein may be configured to monitor and track an extended history of temperatures and/or thermal changes of a product. Some embodiments may utilize flexible algorithms to automatically adjust to various environmental conditions and/or Product-specific regulations. Some embodiments may maintain a temporary or permanent log of defined type(s) of events. Some embodiments may be designed to reduce Product wastage, e.g., by displaying warnings, by estimating or measuring an accurate core temperature of the Product, and/or by monitoring multiple “Product Characteristics” in parallel.

As used herein, “Product Characteristics” of a Product may include any characteristic of the Product itself (e.g., temperature, motion (e.g., acceleration), exposure to light, pressure, chemical characteristics (composition, concentration, changes), electromagnetic/electrostatic exposure, or any characteristic of the environment in which the Product is located (e.g., ambient temperature, humidity, light intensity, etc.). Although this disclosure mainly discusses temperature monitoring, it should be understood that the disclosed systems, methods, and devices can similarly be designed for monitoring other Product Characteristic(s) as an alternative, or in addition, to temperature.

A device as disclosed herein for monitoring one or more Product Characteristics of a Product may be referred to herein as a “Product Monitoring Device,” or “PMD.” Some embodiments provide a PMD that may include an innovative electronic monitoring device and optimized processes, workflows and methods as applicable to the field and the particular Product being monitored. In some embodiments, the PMD provides flexible or adjustable functionality through software programmability. In some embodiments, the PMD provides robust functionality, which may significantly improve reliability and accuracy, as compared with certain conventional monitoring devices and techniques.

In some embodiments, the PMD may provide a significant reduction of false negatives and positives through direct and accurate temperature measurements of the respective Product, as compared with compared with certain conventional monitoring devices and techniques. In some embodiments, the PMD may provide an accurate estimation of the actual core temperature of the respective Product. In some embodiments, the PMD may provide or allow for continuous monitoring throughout the lifecycle of a respective product. In some embodiments, the PMD may utilize advanced algorithms that may be adaptable to a wide range of situations and environments. In some embodiments, the PMD may provide an inexpensive insurance against Product wastage with little to no workflow adjustment. In some embodiments, the PMD may include one or more sensors (e.g., temperature sensor(s)) directly inside the Product or a packaging of the Product (e.g., inside a blood bag) for direct measurements of the core temperature of the respective Product.

In addition to the foregoing, the PMD may also benefit or improve one or more aspects of existing workflow processes for managing temperature-sensitive products. For example, in some embodiments, the PMD may provide fully automatic device activation. In some embodiments, the PMD may have a flexible and inexpensive design that can be reusable or disposable. In some embodiments, the PMD may be configured to log and/or wirelessly transmit temperature or other Product Characteristic data for statistical analysis and/or remote monitoring. In some embodiments, the PMD may include on-board non-volatile memory to store data and allow for subsequent analysis. In some embodiments, the PMD may displaying specific status notifications and/or warnings, e.g., via a display unit (e.g., an LCD or LED(s)) integrated in the PMD. In some embodiments, the PMD may execute customized algorithms to meet relevant standards for a respective type of Product as defined by government, hospital, and/or other relevant regulations. In some embodiments, the PMD may monitor Products according to a multi-state protocol and provide multi-state notifications, which may reflect the health, quality, usefulness, or effectiveness of the monitored products.

Some embodiments provide a temperature measurement device for monitoring the temperature of a temperature-sensitive product, e.g., a blood product. The temperature measurement device may include multiple different types of temperature sensors, e.g., it may include 1 or any combination of the following types of temperature sensors: (a) a product-interfacing temperature sensor in thermal contact with the product, (b) an on-chip temperature sensor of a microprocessor or microcontroller, or (c) an ambient temperature sensor configured to measure an ambient temperature external to the product. The temperature measurement device may further include a processor configured to execute instructions to receive sensor signals from the multiple types of temperature sensors on the device, determine a product temperature of the product based at least on signals from the multiple temperature sensors, compare the determined product temperature with one or more threshold values; and determine whether to generate a notification based on the results of the comparison.

One embodiment provides a Product Monitoring Device (PMD) for monitoring a temperature of a Product, the PMD comprising: at least one product-interfacing temperature sensor in thermal contact with the product and configured to generate first temperature sensor signals indicating a temperature associated with the product; at least one ambient temperature sensor configured to generate second temperature sensor signals indicating a temperature of an ambient environment in which the product is located; a processor; and computer instructions stored in non-volatile computer-readable media. The computer instructions may be executable by the processor to: receive the first sensor signals generated by the at least one product-interfacing temperature sensor; receive the second sensor signals generated by the at least one ambient temperature sensor; determine a product temperature of the product based at least on (a) the first sensor signals generated by the at least one product-interfacing temperature sensor and (b) the second sensor signals generated by the at least one ambient temperature sensor; identify the existence of a notification condition based at least one the determined product temperature; and generate a notification in response to identifying the existence of a notification condition.

In one embodiment, the product comprises a liquid or gas contained in a product enclosure.

In one embodiment, the product comprises a blood pack or other blood product.

In one embodiment, the determined product temperature comprises an estimated core temperature value of the product.

In one embodiment, identifying the existence of a notification condition based at least one the determined product temperature comprises comparing the determined product temperature to at least one threshold value.

In one embodiment, identifying the existence of a notification condition based at least one the determined product temperature comprises calculating a predicted product temperature associated with a defined future time.

In one embodiment, identifying the existence of a notification condition based at least one the determined product temperature comprises calculating a time-based trend in the determined product temperature; and calculating a time until the time-based trend in the determined product temperature crosses a threshold temperature value.

In one embodiment, identifying the existence of a notification condition based at least one the determined product temperature further comprises comparing the calculated time until the time-based trend in the determined product temperature crosses the threshold temperature value to a time threshold value; and generating a notification based on a result of the comparison of the calculated time until the time-based trend in the determined product temperature crosses the threshold temperature value to the time threshold value.

In one embodiment, identifying the existence of a notification condition based at least one the determined product temperature comprises calculating a rate of change in the determined product temperature; comparing the rate of change in the determined product temperature to a threshold rate of change; and generating a notification in response to determining that the rate of change in the determined product temperature exceeds the threshold rate of change.

In one embodiment, the at least one product-interfacing temperature sensor comprises at least one temperature sensor configured to measure a surface temperature of the product.

In one embodiment, the at least one product-interfacing temperature sensor comprises at least one temperature sensor arranged within an enclosure of the product or within an interior volume of the product.

In one embodiment, the device further comprises at least one orientation sensor configured to generate orientation sensor signals indicating a physical orientation of the product; and wherein the computer instructions are executable to determine the product temperature of the product based at least on (a) the first sensor signals generated by the at least one product-interfacing temperature sensor, (b) the second sensor signals generated by the at least one ambient temperature sensor, and (c) orientation sensor signals generated by the at least one orientation sensor.

In one embodiment, the computer instructions are executable to execute a temperature calculation algorithm to determine the product temperature of the product based at least on (a) the first sensor signals generated by the at least one product-interfacing temperature sensor, (b) the second sensor signals generated by the at least one ambient temperature sensor; the device further includes at least one orientation sensor configured to generate orientation sensor signals indicating a physical orientation of the product; and the computer instructions are further executable to automatically adjust the temperature calculation algorithm based on the orientation sensor signals generated by the at least one orientation sensor.

In one embodiment, the computer instructions are executable to determine the product temperature of the product based at least on (a) the first sensor signals generated by the at least one product-interfacing temperature sensor, (b) the second sensor signals generated by the at least one ambient temperature sensor, and (c) a mass or volume of the product.

Another embodiment provides a PMD for monitoring a temperature of a product, the PMD comprising a microcontroller (MCU) comprising an MCU on-chip temperature sensor configured to generate first sensor signals; a product-interfacing temperature sensor in thermal contact with the product and configured to generate second sensor signals; and computer instructions stored in non-volatile computer-readable media. The computer instructions may be executable by a processor to: receive the first sensor signals generated by the MCU on-chip temperature sensor; receive the second sensor signals generated by the product-interfacing temperature sensor; determine a product temperature of the product based on at least one of (a) the first sensor signals generated by the MCU on-chip temperature sensor or (b) the second sensor signals generated by the product-interfacing temperature sensor; identify the existence of a notification condition based at least one the determined product temperature; and generate a notification in response to identifying the existence of a notification condition.

In one embodiment, the processor is provided on the MCU.

In one embodiment, the processor is separate from the MCU.

In one embodiment, the device further comprises an ambient temperature sensor configured to generate third sensor signals indicative of an external environment of the product.

In one embodiment, the device further comprises at least one orientation sensor configured to generate orientation sensor signals indicating a physical orientation of the product; and wherein the computer instructions are executable to determine the product temperature of the product based at least on (a) at least one of the first sensor signals generated by the MCU on-chip temperature sensor or the second sensor signals generated by the product-interfacing temperature sensor, and (b) orientation sensor signals generated by the at least one orientation sensor.

Another embodiment provides a method for monitoring a temperature of a product, the method comprising: receiving, at a processing unit, the first sensor signals generated by the product temperature sensor; receiving, at the processing unit, the second sensor signals generated by the ambient temperature sensor; determining, by the processing unit, a predicted product temperature of the product based at least on (a) the first sensor signals and (b) the second sensor signals, the predicted product temperature indicating a predicted temperature of the product at a future time; comparing, by the processing unit, the predicted product temperature with one or more threshold values; and determining, by the processing unit, based on the comparison, whether to generate an alert signal.

Another embodiment provides a PMD for monitoring a temperature of a product, the PMD comprising: at least one temperature sensor configured to generate temperature sensor signals associated with the product; a processor; and computer instructions stored in non-volatile computer-readable media and executable by the processor to: receive the temperature sensor signals generated by the at least one temperature sensor; determine a product temperature of the product based at least on the temperature sensor signals; calculate a time-based trend in the product temperature; calculate, based at least on the time-based trend in the product temperature, a predicted time at which the product temperature will exceed a threshold temperature value; and generating a notification based on the predicted time at which the product temperature will exceed a threshold temperature value.

In one embodiment, generating a notification based on the predicted time at which the product temperature will exceed a threshold temperature value comprises comparing a time until the predicted time to a time threshold value; and generating a warning notification in response to determining that the time until the predicted time is less than the time threshold value.

In one embodiment, the device implements dynamic wastage thresholds that are closely correlated to or based on existing product regulations, which may require not only the breached temperature threshold but also the time spend above/below certain threshold temperatures.

In one embodiment, the device is configured to generate alerts about impending product wastage based not only on predicted temperature changes but also the exposure time to those temperatures as specified in the guidelines for the specific product of interest and the place (e.g., country or state regulations) where the product is located. The PMD may trigger alerts based on amount of time within and outside of a defined temperature range, based on a magnitude of temperature deviation, based on the product life cycle, or based on any other relevant parameters or events.

In one embodiment, the device implements sensor technology and/or algorithms that enable monitoring of blood products in the context of dynamic wastage thresholds. For example, the device may trigger alerts based on a combination of time out of temperature range, magnitude of temperature deviation, and product life cycle, etc.

In one embodiment, the device is configured to ignore abrupt transient temperature changes, that could be caused by brief contact with warm external objects surfaces. For example, the device may identify and ignore a touch from a hand that could cause a false positive.

Some embodiments provide systems and methods that enable extreme low power and dynamically adjust sensor sampling frequency and active duty cycle based on the detected environmental conditions.

In one embodiment, the PMD may be automatically activated using a simple mechanism such as removal from light-blocking packaging, removal of the adhesive backing or exposure to specific temperatures for specific duration of time.

In one embodiment, the PMD may be automatically activated based on detection of a capacitance change caused by the PMD being attached to or detecting the product in proximity of the PMD. (e.g., the PMD may activate itself upon determining that it is attached to a blood product, e.g., based on capacitance sensor signals).

In one embodiment, the device is configured to automatically initiate monitoring once a blood product is donated. For example, tags can be attached to empty blood bags and remain in low-power sleep mode. Once blood is donated and blood enters the bag, the capacitance change induced by the fluid can wake device and initiate continuous monitoring. Alternatively, the PMD may be triggered by an abrupt temperature change to exit low-power sleep mode. The temperature may initially be high (e.g., 37° C.) but should decrease rapidly once donation is complete and the product is placed in a refrigerator (the temperature should then remain between 4-10° C. for the remainder of the product life cycle).

In one embodiment, the PMD may be configured to monitor the contents of a product using a capacitive sensor. For example, a capacitive sensor can be used to verify that there is enough liquid in the bag to be monitored. If the sensor determines that there is not enough liquid, the PMD will stop monitoring and/or displaying status alerts.

In one embodiment, the PMD may be configured to be activated or controlled via button push(es) or capacitive touch patterns. Different touches or patterns may activate different modes such as the configuration of the PMD for the type of product to be monitored or may prevent the device from continuing to display or sound alerts when they are not necessary.

In one embodiment, the PMD may provide an easy-to-understand interface, e.g., based on LED colors and/or blinking patterns, which may be implemented with low power and may be sensitive to the unique needs of the particular application.

In one embodiment, PMDs designed for different types of products may be provided with distinguishable PMD labels, shapes or colors that may be representative of the Products they are configured to monitor. Each different PMD may have unique temperature thresholds, timing thresholds and/or alert types depending on the specific product being monitored.

In some embodiments, the PMD may provide multiple sensors on the surface or inside the product housing to more accurately and uniformly measure the temperature of the product.

In some embodiments, the PMD may be configured to directly measure the core temperature of the product with one or more sensors by embedding the PMD directly inside the product packaging.

In one embodiment, the PMD may utilize multiple temperature sensors placed uniformly inside the product packaging to accurately measure and average the core temperature of the product.

In one embodiment, the PMD may be configured for reuse through a built-in reset mechanism (e.g., a push button pattern) and/or through the use of a detachable (e.g., snappable) mounting attachment.

In one embodiment, the PMD may be configured to monitor a product using a lab-on-chip, e.g., to monitor biochemical/microfluidics parameters of a product.

### BRIEF DESCRIPTION OF THE FIGURES

FIG. 1 shows an example product monitoring device (PMD) for monitoring a Product, e.g., a blood product or other type of product, according to one embodiment;

FIG. 2 illustrates example types of sensors that may be provided in a PMD;

FIG. 3 illustrates an example PMD memory device and example types of data that may be stored in such memory device;

FIG. 4 illustrates an example PMD operational flow, according to an example embodiment;

FIG. 5 illustrates an example process flow executed by a PMD for using a static resistive model, according to an example embodiment;

FIG. 6 illustrates an example PMD attached to an example Product (e.g., a blood product), wherein the PMD is configured to output information to a user, e.g., hospital personnel or a blood bank technician;

FIG. 7 illustrates a flowchart of an example algorithm executable by a PMD to determine an impending Product wastage condition, according to an example embodiment;

FIG. 8 illustrates a flowchart of an example algorithm executable by a PMD to determine a temperature violation, according to an example embodiment;

FIG. 9 illustrates a flowchart of an example algorithm executable by a PMD to determine an impending Product life expiration condition, according to an example embodiment;

FIG. 10 illustrates a flowchart of an example algorithm executable by a PMD to monitor a Product, according to an example embodiment;

FIG. 11 illustrates a flowchart of an example algorithm executable by a PMD to determine a state change of the Product, according to an example embodiment;

FIG. 12 illustrates an example algorithm executable by a PMD for outputting Product state information upon user request, according to an example embodiment;

FIG. 13 illustrates an example algorithm executable by a PMD for controlling enabling/disabling the output of notifications via one or more output devices, according to an example embodiment;

FIG. 14 illustrates an example algorithm executable by a PMD for providing redundant temperature monitoring, according to certain embodiments;

FIG. 15 illustrates an example algorithm executable by a PMD for calculating a Product temperature as a weighted average of the temperature readings of an MCU internal on-chip temperature sensor and a separate product-interfacing temperature sensor, according to an example embodiment;

FIG. 16 illustrates an example algorithm executable by a PMD to monitor a Product (e.g., to monitor the temperature of a blood product), according to some embodiments;

FIG. 17 illustrates an example algorithm executable by a PMD to monitor a Product to detect an occurred or impending Product wastage event, according to one embodiment;

FIG. 18 illustrates an example algorithm executable by a PMD to monitor accumulated or “lifetime” heating of Product, according to an example embodiment;

FIG. 19 illustrates an example algorithm executable by a PMD for selectively implementing different core temperature calculation algorithms based on a Product orientation determined using at least one orientation sensor, according to an example embodiment;

FIG. 20 illustrates an example algorithm executable by a PMD to analyze the validity of sensor data generated by one or more PMD sensors;

FIG. 21 illustrates an example algorithm executable by a PMD for monitoring a Product to identify a current or imminent Product wastage event, according to an example embodiment;

FIG. 22 illustrates an example temperature curve extending from the ambient environment and through the Product core, in a situation in which the Product is thermally influenced only/substantially only by convection;

FIG. 23 illustrates an example temperature curve for a situation in which the Product is resting on a surface of solid structure (e.g., shelf or table), according to an example embodiment;

FIG. 24 illustrates an example algorithm executable by a PMD for calculating a core temperature of a Product using a thermal model, according to one embodiment;

FIG. 25 illustrates an example static thermal model to describe the thermodynamic system of an example Product;

FIG. 26 illustrates another example static thermal model to describe the thermodynamic system of an example Product;

FIG. 27 illustrates an example algorithm executable by a PMD to monitor the core temperate of a Product using on at least one product-interfacing temperature sensor and at least one ambient temperature sensor, according to an example embodiment;

FIGS. 28A-28E illustrate an example prototype of a PMD;

FIG. 29 illustrates an example debug/programming device configured to receive a PMD assembly via a plug-in type connection, to provide a physically interfaces for communicating data, signals, and/or power to and/or from PMD assembly, according to an example embodiment;

FIGS. 30A-30E illustrate various views of another example PMD for monitoring the temperature of a Product, e.g., a blood product, according to example embodiments;

FIG. 31 illustrates an external side view of an example PMD attached to a blood pack, according to one embodiment;

FIG. 32 illustrates an example blood pack (filled with saline) with a PMD enclosure of an example PMD attached to an outer surface of the blood pack, according to one embodiment;

FIGS. 33A-33D illustrate example PMDs that include at least one internal Product temperature sensor located inside the Product, e.g., inside a blood bag of a blood product, according to example embodiments; and

FIGS. 34A and 34B illustrate example embodiments of a PMD packaging including one or more recesses for providing increased exposure of one or more temperature sensors to the ambient environment.

### DETAILED DESCRIPTION

Example Product Monitoring Device (PMD)

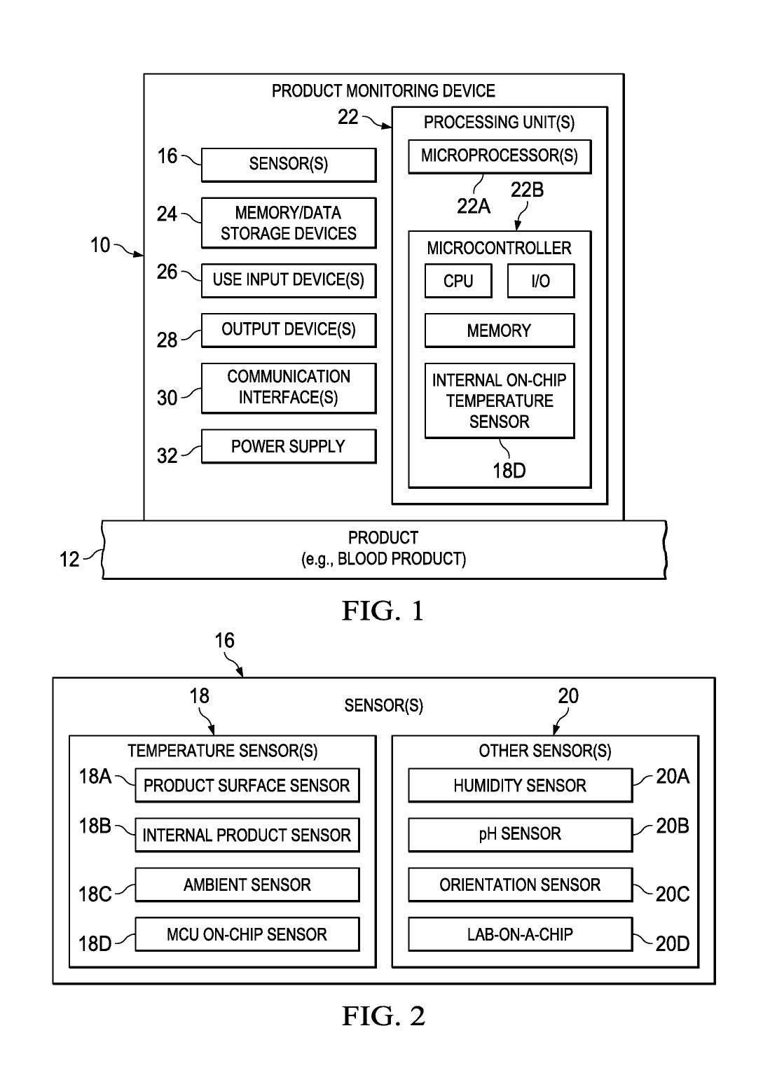

FIG. 1 shows an example product monitoring device (PMD) 10 for monitoring a Product 12, e.g., a blood product or other type of product, according to one embodiment. PMD 10 may include any suitable hardware, software, firmware, and other components for performing any of the disclosed functionality. For example, as shown in FIG. 1, example PMD 10 may include, sensor(s) 16 for detecting one or more Product Characteristics, processing unit(s) 22 for processing data, memory or data storage unit(s) 24 for storing data, user input interface(s) 26 for receiving commands or other input from a user, output device(s) 28 for outputting notifications or other information to a user, wireless and/or wired communication interface(s) 30 for receiving data at PMD 10 and/or transmitting data from PMD 10 a power supply 32 for providing power to PMD 10, and/or any other components for performing any functionality disclosed herein or any logically related functionality. The various components of example PMD 10 are discussed in further detail below.

Processing Unit(s)

PMD 10 may include one or more processing units 22, which may include one or more microprocessors, microcontrollers (MCUs), and/or other type(s) of digital processing units.

A microcontroller typically includes memory, programmable input/output peripherals, and a processor (CPU) integrated on a single chip. In embodiments in which PMD 10 includes one or more MCUs 22, each MCU 22 may include any suitable type of CPU, for example, a MSP430, ARM Cortex M0, Intel MCS-51(8051), Atmel AVR, Microchip PIC, or any other suitable CPU. Further, in some embodiments, each MCU 22 may be an ultra low power (ULP) microcontroller (MCU), for example, a ULP microcontroller that implements a sleep mode with a wake up timer that consumes <10 μA, and an active mode that consumes up to 10 mA. Each MCU 22 may also include an analog to digital converter (ADC) for processing data from one or more analog sensors 16.

In some embodiments, the processing unit 22 of the PMD 10 are operable to execute relevant software or computer instructions, e.g., in the form of logic-based algorithms, to control the operation of the various aspects of PMD 10, e.g., collecting data from sensor(s) 16, processing/analyzing the collected sensor data (e.g., to determine a current or predicted state of the Product), storing the sensor data or data derived from the sensor data (e.g., Product state information) in data storage unit(s) 24, generating alert notifications, transferring data and/or alert notifications via the wired or wireless communication interface(s) 30, receiving user input via user input interface(s) 26 and processing such user input, and/or controlling output device(s) 28 to output notifications or other information to a user (e.g., via LEDs, sound, visual messages, etc.).

PMD Sensor(s)

PMD 10 may include any one or more sensor(s) 16 suitable for detecting one or more Product Characteristics, e.g., one or more temperature sensors and/or other type(s) of sensors.

FIG. 2 illustrates some example types of sensors 16 that may be provided in a PMD 10. As shown, PMD 10 may include one or more temperature sensors 18 and/or one or more other types of sensors 20. Temperature sensors 18 may include (a) one or more Product surface sensors 18A configured to interface with and detect a surface temperature of a Product or Product packaging, (b) one or more internal Product sensors 18B configured to be suspended or otherwise located within a Product or Product packaging (e.g., sensor(s) suspended or located inside a blood pack) for directly measuring an internal or core temperature of the Product, (c) one or more ambient sensors 18C configured to measure an ambient temperature of the environment in which the Product is located, (d) one or more microcontroller (MCU) internal on-chip sensors 18D, integrated in one or more MCUs 22B provided in the PMD, and/or (e) any other type of temperature sensor. Each temperature sensor 18 may comprise a thermistor, resistive temperature detector (RTD), thermocouple, solid state junction temperature sensor, optical temperature sensor, integrated circuit (IC) temperature transducer, other digital temperature IC, and/or any other type of temperature sensor.

As shown in FIG. 2, PMD 10 may also include one or more other sensors 20, such as one or more humidity sensors 20A, pH sensor 20B, orientation sensor 20C, “lab-on-a-chip”/microfluidic or other sensor capturing bio- or chemical characteristics 20D, and/or any other type(s) of sensors. Orientation sensors 20C may include one or more types of sensors configured to detect a physical orientation of PMD 10, for example one or more accelerometers, tilt sensors, magnetometers, altimeters, etc. A lab-on-a-chip (LOC) 20D is a device that integrates one or several lab functions on a single integrated circuit (chip), and may be operable to provide chemical or biological composition monitoring of a Product, e.g., to determine concentrations of one or more substances or biological entities present in the Product. In some embodiments, one or more sensors 16 of the PMD 10 may have direct contact with the Product, may have a fixed location proximate the Product, or may be suspended directly inside the Product or Product packaging. Each sensor(s) 16 may either be thermally coupled or partially or fully thermally isolated from the Product. For example, PMD 10 may include a Product-interfacing surface temperature sensor in thermal contact with the Product and/or an ambient temperature sensor located proximate, but not in contact with, the Product. The data from the PMD sensor(s) 16 may be combined in a novel way to be integrated into software algorithms to provide improved accuracy, reliability and/or functionality, e.g., as described below.

In some embodiments, one or more sensors 16 may either be mounted directly to the PMD 10 (surface mounted SMT) or may extend or protrude out of the PMD 10 to achieve more direct thermal contact and/or improve thermal isolation between the PMD 10 and sensor 16. Finally, a thermal sensor 16 may be secured in place, e.g., by glue or thermal paste, to improve heat transfer between temperature sensor(s) 18 and a monitored Product.

Microcontroller (MCU) Internal On-Chip Temperature Sensor

Microcontrollers (MCUs) typically include an internal on-chip junction sensor (temperature sensor) that monitors the MCU for the purpose of preventing thermal runaway in the chip. Embodiments of PMD 10 that include an MCU 22B (or multiple MCUs 22B) utilize this internal on-chip temperature sensor of the MCU, referred to herein as the MCU internal on-chip temperature sensor 20D, as one input (either alone or in combination with other input(s), e.g., measurements from one or more other sensors 16 of PMD 10) for determining a temperature of the Product (or a temperature having a known or expected correlation with a temperature of the Product).

This novel use of the MCU internal on-chip temperature sensor 18D is based on a correlation between the MCU chip's internal temperature and the temperature external to the MCU chip/package This correlation may be particularly defined or predictable where the system/MCU operates according to a low active duty cycle (e.g., below 1%, running 10 ms and sleeping 10 seconds), such that thermal heat generated in the chip itself is negligible and escapes the MCU package much faster than it is generated. The result is that the temperature inside the MCU package and on the silicon die may be largely defined by, and thus approximately equal to, the temperature outside the MCU. PMD 10 may thus measure the temperature of the Product (or a temperature having a known or expected correlation with the Product temperature) either solely using the MCU internal on-chip temperature sensor 18D, or in combination with one or more other sensors 16 of the PMD 10, e.g., by combining the readings of multiple temperature sensors 18 (e.g., by averaging or other mathematical combination), or by using the MCU internal on-chip temperature sensor 18D to calibrate, correct, error-check, feasibility-check, and/or otherwise evaluate the measurements of other sensor(s) 16 of the PMD 10. In some embodiments, the MCU internal on-chip temperature sensor 18D may also be used to calibrate or verify other temperature sensors 18 throughout the lifetime of the PMD 10. The MCU internal on-chip temperature sensor 18D may be used alone or in combination with other sensor(s) 16 to determine the temperature of a specific portion of the Product or a temperature directly affecting the temperature of the Product (e.g., surface temperature or ambient temperature). Some embodiments of the PMD 10 correlate the temperature detected by the MCU internal on-chip sensor 18D with the ambient temperature, surface temperature, or other temperature(s), and thus use the MCU internal on-chip sensor 18 for determining such temperature(s).

Use of Multiple Sensors

In some embodiments, PMD 10 may use multiple sensors 16 of the same type or different types that measure the same Product Characteristic (e.g., temperature) or different Product Characteristics (e.g., temperature, humidity, and orientation). In some embodiments, PMD 10 may include multiple sensors 16 for the purpose of redundancy to increase reliability and accuracy of the relevant Product Characteristic measurements. As another example, PMD 10 may use multiple sensors to facilitate algorithm(s) executed by the PMD 10 (e.g., algorithms 52 shown in FIG. 2) to derive certain Product Characteristic(s) and/or environment characteristic(s) or state(s) that may not be available for direct measurement. PMD 10 may include hardware that conditions the sensor data collected by each of these sensors 16, and software/algorithms that further process or analyze the conditioned sensor data. Using this combination of sensors and software/hardware may provide more accurate and robust temperature measurement and analysis as compared with certain conventional sensor products, and may also allow for useful predictive analysis, e.g., to predict a temperature violation and generate an alert prior to the occurrence of the violation.

For example, some embodiments of PMD 10 use an MCU internal on-chip temperature sensor 18D and/or one or more other temperature sensors 18 to increase accuracy and robustness of measuring and/or calculating a temperature (e.g., a volume/core temperature) of the Product. As noted above, some embodiments use a combination of sensors 16 to measure and/or calculate a temperature (e.g., a volume/core temperature) of the Product. For example, in some embodiments PMD 10 collects and analyzes data from (a) an MCU internal on-chip temperature sensor 18D, (b) at least one product-interfacing temperature sensor 18A in thermal contact with the Product, which detects a temperature of, or closely correlated with, a surface temperature of the Product, and (c) at least one ambient temperature sensor 18C configured to measure an ambient temperature of the Product's environment.

One or more product-interfacing temperature sensors 18A in thermal contact with the Product may provide dynamic temperature characteristics of the Product. In some embodiment, each product-interfacing temperature sensor(s) 18A may be arranged to directly contact a surface of the Product, or may be separated from the Product by a thermally-conductive barrier, e.g., a thin thermally conductive film on the surface of a bag or other packaging that contains the Product. In some embodiments, product-interfacing temperature sensor(s) 18A can also detect/monitor a localized temperature profile, and together with one or more other sensors 16 of PMD 10, allow the PMD 10 to determine various characteristics of a temperature profile of the Product.

Some embodiments of PMD 10 include one or more ambient temperature sensors 18C to supply additional data to the temperature analysis algorithms 52 executed by the PMD 10, e.g., to increase the accuracy or reliability of a determined current temperature of the Product and/or to calculate a predicted (future) temperature of the Product.

PMD 10 include one or more temperature sensors 18, e.g., any of the sensors discussed above, to measure and record a surface temperature of a Product. In some embodiments, PMD 10 may use such surface temperature data to calculate an estimated core temperature of the Product, e.g., as discussed in more detail below.

Memory/Data Storage Unit(s)

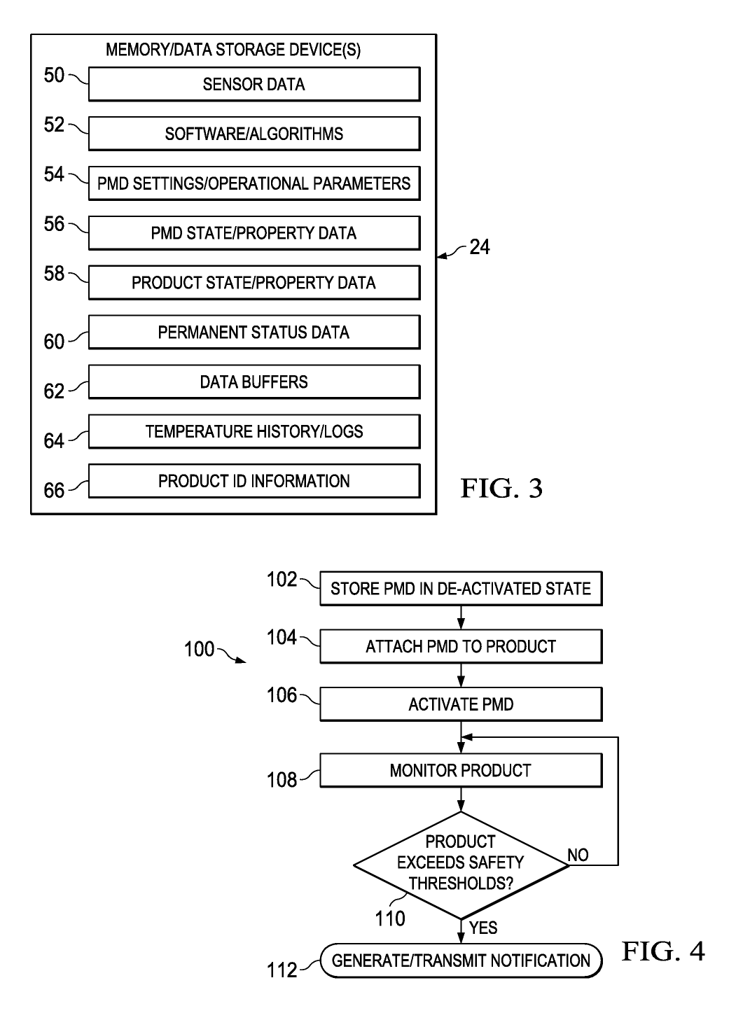

Data storage unit(s) 24 may include one or more volatile and/or non-volatile memory devices for storing various types of information. FIG. 3 illustrates an example memory device 24 For example, as shown in FIG. 3, data storage unit(s) may store any of the following types of data:
sensor data 50 (e.g., data read from sensor(s) 16),
algorithms and/or other software/processor instructions 52 (e.g., for executing the functions of PMD 10, analyzing sensor data, determining temperature values, identifying state changes or notification conditions, generating notifications or alarms, etc.),
PMD settings or operational parameters 54.
PMD state and/or property data 56,
Product state and/or property data 58,
critical permanent status parameters 60 (e.g., Product is good/bad) and any other flags or warnings that would need to be stored across possible PMD resets,
temporary data buffers 62,
temperature history or logs 64,
Product identification information 66, and/or
any other types of data related to PMD 10, the Product, the environment of the Product, etc.

Although FIG. 3 illustrates an example data storage device 24, different types of data may be stored on multiple different data storage device 24 provided in PMD 10. For example, PMD 10 may store selected types of data in a volatile memory device and/or other types of data in a non-volatile memory device.

Such non-volatile memory devices may include one or more persistent memory modules based on Flash Memory, EEPROM, FRAM, MRAM, and/or ReRAM, for example. Volatile memory devices may include SRAM and/or DRAM, for example. Some of the aforementioned technologies such as FRAM and MRAM require very little energy for operation and may thus be particularly suitable for use in certain embodiments of PMD 10, e.g., embodiments of PMD 10 that utilize energy from the surrounding environment through the use of harvesting techniques.

The data storage unit(s) 24 of PMD 10 may provide temporary or long-term storage of collected sensor data (from sensor(s) 16), which PMD 10 may analyze by executing one or more software algorithms to assess the temperature or state of the Product and/or predict future temperature dynamics. Some embodiments or configurations of PMD 10 may log and preserve raw sensor data received from sensor(s) 16 in memory for future analysis of such data. Other embodiments or configurations may process the raw sensor data and store the results of such processing (e.g., changes in Product state or instances of other defined events, e.g., detection of temperatures that cross a defined threshold, etc.) and erase or allow overwriting of the raw sensor data, to thereby reduce data storage needs.

Power Supply

PMD 10 may include any suitable type or types of power supply 32 for powering the operation of the PMD. In some embodiments, PMD 10 may include one or more batteries for providing power to the PMD. One embodiment of PMD 10 includes a CR2032 battery as a power supply 32, which is estimated to cover the typical life-cycle of a Product. In other embodiments, PMD 10 may include components or devices for harvesting and storing energy from external sources through light, radio waves, physical movement or other means. Harvested energy can be stored in any suitable energy storage device, e.g., supercapacitors, rechargeable batteries (e.g., Li-ion), or a combination thereof.

User Interfaces: Input/Output

PMD 10 may include any suitable user input interfaces or devices 26 configured to receive user input and any suitable output devices 28 configured to output data or notifications to a user.

User input interfaces 26 may include, for example, one or more buttons, switches, dials, sliders, capacitive sensors, any other sensors for detecting human input, e.g., an accelerometer, a communication interface or other physical user interfaces.

Output devices 28 may include, for example, one or more visual indicators (e.g., one or more LEDs and/or LCD screens or other display screens); audible output devices (e.g., speakers or sound buzzers); tactile feedback output devices, permanent irreversible indicators, (e.g., e-Ink, fuses, etc.), a wired or wireless interface for transmitting status, data, and/or notifications to an external device.

Communication Interfaces

In some embodiments, PMD 10 may include wireless and/or wired communication interface(s) 30 for receiving data at PMD 10 and/or transmitting data from PMD 10. Wired communication interfaces may include interfaces according to any of the following communication protocols: USB, UART, SPI, I2C, SMB, JTAG, C2, CAN, LIN, ethernet, PCIe, or any other type(s) of wired communication protocol.

Wireless communication interfaces may include transmitters and/or transceivers, and any associated hardware, software, and/or firmware configured to provide wireless communications according to any of the following communication protocols: RFID, Bluetooth/BLE, Wi-Fi, near field communications (NFC), ZigBee, GSM/CDMA/3G/4G/LTE/5G cellular communication, or any other wireless communication protocol.

In some embodiments, PMD 10 may be configured for wireless communications that enable internet cloud-based or other network-based monitoring of one or more Products. For example, PMD 10 may include any combination of the aforementioned wireless communication interfaces 30 for communicating data regarding the PMD 10 or a respective Product (e.g., detected temperature data, Product state information, Product identification information, alert notifications, PMD's data, etc.) to the internet cloud, server or other network-based system, or other wireless devices, e.g., mobile phones, tablets, smart wearable devices, etc.

In some embodiments, data may be collected from a PMD 10 using an RFID field for transferring power to the PMD 10 and collecting data stored in onboard memory of the PMD 10 while an active RFID field is present. This may allow for an extremely low-power PMD 10 potentially eliminating the need for an actual power from a battery.

PMD Attachment to Product/Packaging

In some embodiments, PMD 10 may be attached to a Product, may be secured on or near a Product or suspended or otherwise located inside a Product (e.g., inside a blood bag). Some example ways of attaching the PMD 10 to a Product are discussed as follows. In some embodiments, PMD 10 may be secured in place with an adhesive material, e.g., on a back surface of the device. The adhesive material may include features to reduce or minimize thermal resistance between the temperature sensor(s) 18 of the PMD 10 and the Product, to thereby improve responsiveness and accuracy of the temperature measurement. For example, the temperature sensor(s) 18 may be arranged in direct contact with the PMD housing. The housing, at least in a region between the temperature sensor(s) 18 and the Product, may be formed from one or more materials with high thermal conductivity, e.g., thin metal or tape sheets.

In other embodiments, PMD 10 may be configured such that it does not directly contact the Product, but is physically associated with the Product in a manner that allows the PMD 10 to effectively monitor the Product Characteristic(s) of the Product. For example, the PMD 10 may be arranged at a known, constant location relative to the Product. In some embodiments, a PMD 10 can be incorporated into a medication bottle cap, or in shipments of temperature-sensitive Product(s).

In some embodiments, PMD 10 may be suspended or otherwise located directly within the Product (e.g., a liquid or gaseous Product) or inside the Product packaging. PMD 10 may have a housing that defines a sealed enclosure to avoid ingress of liquids and provide a low permeability to water, which may be advantageous and allow the PMD and/or one or more sensor(s) 16 of the PMD to be directly suspended or otherwise located inside a liquid or gaseous Product. The PMD 10 may also be directly embedded into the Product packaging, which may allow for PMD sensor(s) 16 to be in direct contact with the inner volume or core of the Product. Some embodiments may include multiple sensors 16 suspended or otherwise located inside the Product/Product packaging to increase accuracy and/or thoroughness of the measurement of the Product core (e.g., core temperature). In some embodiments, a desiccant may be arranged in the PMD enclosure to avoid/reduce condensation formation within the PMD.

In some embodiments, PMD 10 may be clipped onto a Product using a one-time or multi-use clipping mechanism that may ensure that the PMD 10 is securely attached to the Product with a mechanical clip or inserted into a reusable mechanical mount. In some embodiments the Product may be manufactured with a secure mounting mechanism and the PMD 10 may be inserted into such mechanism and mechanically secured to the Product. In one embodiment, PMD 10 may be reusable and rely on a multi-use mechanical mount that may be attached to the Product prior to activation of the PMD and removed and recycled after some duration, e.g., after completion of the relevant Product monitoring cycle (e.g., once the Product has been used, has spoiled, etc.).

PMDs 10 can have different tags (shape, size, color, feel, lettering, indicia, etc.) designed for different Products, e.g., different blood products. With respect to blood products, the PMI) tags may be easily distinguishable from each other and may have internal programming designed for a specific type of blood product (PRBC, FFP, platelet, cryo, etc.). Storage parameters may be different depending on the type of Product.

In some embodiments, PMD 10 may include a lab-on-a-chip or similar device that may need to be in direct contact with the product. In this case, the packaging of the product would have an opening designed specifically to fit the lab-on-a-chip and keep the rest of the PMD sealed away from the Product. In some embodiments, PMD 10 may use a lab-on-chip device to sense biochemical, physical, mechanical or any other relevant characteristics of the whole product or parts of the product (e.g., at the scale of a single cell or molecule). Such lab-on-chip device, for example, may rely on microfluidics “microbead filtration method” to sense the level of cells' deformability and stiffness, which mimics the in-vitro environment and provides information on how effective the blood may be if transfused. In another embodiment the lab-on-chip may measure electrical properties of the cells to detect activated leukocytes that may be correlated with the risk of sepsis.

In some embodiments, a PMD 10 including a lab-on-chip may directly filter and remove cells or molecules that are characterized to be unfit for the use. Such PMD 10 may improve the overall quality of the products used and/or save some Product instances from being wasted by cleaning the solution from cells and molecules, which do not meet required standards or specifications. For example, in case of red blood cells, such PMD 10 may filter out activated leukocytes and/or red blood cells that do not pass through deformability and stiffness filters as well as other unwanted molecules.

In some embodiments, a PMD 10 including a lab-on-chip device may be placed inside the Product package (e.g., blood bag) or inside the product delivery system (e.g., in an IV), where it could monitor and filter the Product during use, or in a separate container where the Product may be transferred specifically for monitoring, analysis, filtering and/or cleansing, for example.

PMD Operation

FIG. 4 illustrates a PMD operational flow 100, according to an example embodiment. At 102, PMD 10 may be stored in a de-activated state, waiting for activation At 104, PMD 10 is attached to a Product. PMD 10 is then activated at 106 and starts monitoring Product Characteristic(s) at 108. In some embodiments, after activation at 106, PMD 10 remains in a low power/sleep mode and periodically wakes up from the low power/sleep mode to read data from on-board sensor(s) 16, and keep track of time.

While in the “awake” phase of the monitoring process 108, PMD 10 may perform one or more of the following functions: (a) process sampled sensor data, (b) analyze the sensor data, (c) determine a state of the Product, (d) read user input, if any, received via a user input interface 26, (d) identify and generate output if appropriate (e.g., based on determined Product state and/or in response to user input, e.g., a status query), and (e) store data and other information in memory/data storage device(s) 24. After completing the wake phase, the PMD may go into a low power mode/sleep mode for a defined time period before entering the next wake phase, and repeat this sleep phase/awake phase cycle during the monitoring of the Product. At 110, PMD 10 may determine whether the sensor data collected and analyzed at 108 exceeds any relevant threshold values, and if so, generate and output notification, e.g., by outputting a notification to the user via an output device 26 and/or by communicating a wireless notification signal to a remote system or device, for example.

As used herein, “exceeding” a threshold value includes both (a) a value that is greater than an upper threshold value and (b) a value that is lower than a lower threshold value. Thus, a determined product temperature may exceed an upper threshold value by increasing above such threshold value, and may exceed a lower threshold value by falling below such threshold value. In addition, exceeding a threshold value may or may not include meeting the threshold value exactly, depending on the particular implementation, algorithm specifics, or PMD setting, for example. Thus, for any teaching herein of determining whether a threshold value is exceeded by a calculated value, in one embodiment the threshold value is exceeded if the calculated value exactly meets the threshold value, while in another embodiment the threshold value is exceeded only if the calculated value goes above an upper threshold value or below a lower threshold value.

In some embodiments in which PMD 10 is reusable, the PMD may be reset after use on one Product and reused on another Product by repeating process 100. As a part of such reset, any data stored on the PMD 10 may be transferred via a wired or wireless interface for additional processing.

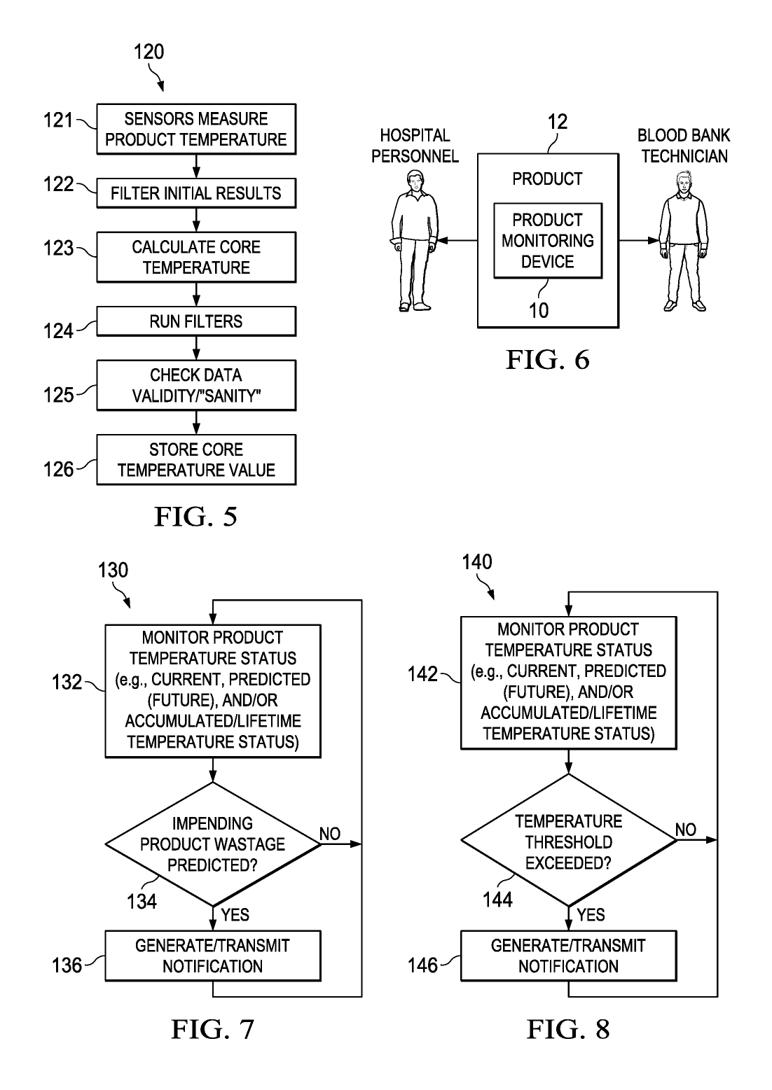

FIG. 5 illustrates an example process flow 600 executed by PMD 10 for using a static resistive model, e.g., as disclosed below, according to an example embodiment. At 602, one or more temperature sensor(s) 18 of PMD 10 may take one or more temperature measurements and store the sensor data. At 604, PMD 10 may filter the sensor data using one or more filtering algorithms (e.g., IIR, FIR, Kalman, etc.) to provide a high frequency noise filtering. The filter weight(s) may be adjusted to match a dynamic rate of change of each sensor, which may help reduce transient error due to varying dynamic rates. At 606, PMD 10 may calculate an estimated core temperature based at least on the stored and filtered temperature measurements, e.g., using any algorithm and/or model disclosure herein. At 608, PMD 10 may execute one or more filtering algorithms (e.g., IIR, FIR, Kalman, etc.) to provide filtering that would simulate the mass and/or volume of the Product. At 610, PMD 10 may check for invalid data results, e.g., by running “sanity checks” to ensure that the estimated core temperature calculated at 606 is sensible, e.g., to ensure that the core temperature is not trending opposite the ambient temperature, or experiencing excessively sudden changes, etc. If the estimated core temperature passes the check at 610, PMD may then store the estimated core temperature value at 612.

PMD Activation

A PMD 10 may be distributed and stored in a de-activated state and may be activated using any suitable user input device 26 and/or in any other suitable manner, e.g., via temperature, light, acoustic, capacitive sensing, voltage sensing, magnetic/electric field sensing (Hall effect), etc. Some examples of PMD activation include any one or a combination of the following:
Mechanical button: PMD 10 may include a mechanical button 26 for activation of the PMD by a single button press or a defined or custom pattern of button presses designed to decrease the likelihood of erroneous or accidental activation.
Capacitive activation: PMD 10 may include a capacitive sensor 20, 26 configured to detect a touch by a user, or a proximity to the user (e.g., a wave by the hand over the device close enough to trigger a change in capacitance) that triggers the PMD 10 to activate itself. Like the mechanical button discussed above, the PMD may specify a defined or custom pattern of touching/movements detected by the capacitive sensor. In some embodiments, the capacitive sensor may be configured to detect the Product, which may trigger the PMD to activate.
Temperature activation: A user may press and hold a finger on a designated area 26 of the PMD for a period of time, which heats a temperature sensor 18 above a predetermined threshold or causes a temperature change greater than a predetermined threshold, which triggers the PMD to activate itself.
Light activation: PMD 10 may include a light sensor 20 and may be packaged such way that very little light penetrates the packaging, or such that the light sensor 20 is covered by non light-permeable material. When the package is opened, or the covered light sensor 20 is exposed to light, the light sensor detects the increased light intensity and the PMD 10 activates itself
Voltage based activation: PMD 10 may include a simple piece of conductive material adhered to two external contacts (one contact arranged to sense a voltage and the other connected to power or ground of the circuit), effectively shorting them. This causes the PMD to detect a voltage high/low (depending on the particular configuration) on the input pin. The removal of the material effectively changes the state at the voltage sensing input from high to low, or vice versa, which the PMD 10 may use as a trigger to activate the device.
Magnetic or Electric field based activation: PMD 10 may include a hall effect or other type of sensor 20 configured to sense a change in magnetic field and activate the device in response. This allows for touchless activation where the PMD 10 may be activated by placing the PMD over a sufficient magnetic field.
Activation upon power source attachment: PMD 10 may be activated by connecting a battery 32 or by removing a barrier blocking the battery or another power 32 source from powering the PMD 10. The removal of the barrier closes a circuit thus allowing the electrical current, to flow through the PMD 10.
Activation upon Product Characteristic change: PMD 10 may start active monitoring of a Product's state after detecting a temperature within a certain range. for example, within a valid storage criteria range. PMD 10 may start monitoring after detecting the presence of a Product based on chemical or physical characteristics detected by sensor(s) 16. In addition, if certain thermodynamic properties of the Product are known, PMD 10 may be programmed to determine whether the Product is full, empty, or at any point in between. Through monitoring of thermodynamic properties it may be possible to determine when there is no Product left in the bag or other packaging and consequently detect when the Product has been filled and used/emptied. PMD 10 may also include an accelerometer 20 to augment the previous methods, e.g., wherein PMD 10 may apply spatial product positioning rules to determine the status of the Product.

Alerts and Other Feedback/Device Output

During operation, PMD 10 attached to a Product may determine the state of the Product, which may include a current state and/or a predicted future state, based on collected sensor data from PMD sensor(s) 16. PMD 10 may provide feedback using any available output interface or device 28, e.g., visual output device(s) such as an LCD display, LED(s), multi-color LED(s), e-ink, etc.; sound output device(s), tactile feedback device(s), etc. Example output device(s) 28 are discussed below in the user interface section.

PMD 10 may output notifications or information to indicate a state of the Product and/or the PMD 10, and/or any other critical or user-defined information available to PMD 10. Such output signals may include, for example:
Product state information: some examples include: good/usable, bad/expired/unusable, approaching a threshold (future violation predicted), health percentage, product age, time till expiration or violation, time passed (e.g., number of hours) in expiration or violation state, product fill level (e.g., blood bag not fully or fully filled or percentage of the level fill), product characteristics parameters, excessive or insufficient levels of biological or chemical substances, etc.
PMD state information: some examples include: active, inactive, functional, product detected, malfunction occurred, low battery, feature set, connectivity status, components status, collected data, etc.

Triggers for such data output may be automated (e.g., programmed in the PMD), e.g., upon the presence of a defined triggering condition for communicating information to the user, or data output may be user-initiated based on input from the user via a user interface 26. Examples of such data output triggers include:
Receive user input requesting information from PMD 10.
Automated: PMD 10 generates a warning in in response to relevant Product Characteristic(s) approaching defined threshold value(s), e.g., informing the user to take an action to prevent Product wastage.
Automated: PMD 10 generates a Product state or PMD state indication.

Additionally, in some embodiments, output notifications or signals may be enabled or disabled based on relevant operating conditions. PMD 10 may be configured to temporarily disable and adjust certain outputs to preserve power if it determines that a particular output is not necessary. Some examples may include:
PMD 10 may determine that the Product is in storage (e.g., in a refrigerator) as determined based on relevant sensor data from sensor(s) 16 (e.g., low light or consistently cold ambient temperature) and an output notification is not accessible (e.g., not visible) or useful to the user. In such embodiments, the PMD 10 may disable any output notifications.
PMD 10 may be configured to output certain product states or other output signals only in response to a user input query, and not automatically triggered by PMD itself, to preserve battery life.
PMD 10 may be configured to disable some or most output triggers disabled, and enable only defined critical output triggers, e.g., a warning of an approaching product wastage state.
PMD 10 may make adjustments to the output signals (e.g., enabling and disabling various outputs) based on the current PMD state, e.g., battery life level.

FIG. 6 illustrates an example PMD 10 attached to an example Product 12 (e.g., a blood product), wherein the PMD 10 is configured to output information, e.g., Product state information, alert information, etc. to a user, e.g., hospital personnel or a blood bank technician.

Some example typical use cases for PMD 10 may include:
In case where the Product is left outside of a cooler for too long, PMD 10 may activate a warning LED 28 to alert a nearby person.
PMD 10 may provide an output signal as a feedback upon activation by a user.
If PMD 10 determines a current alert-triggering state, e.g., a temperature violation state in which the detected temperature of the Product has crossed a defined threshold value, PMD 10 may generate an alert signal to activate corresponding LED(s) 28.
PMD 10 may be configured to predict a future state of the Product, e.g., based on determined trends in the detected Product temperature and/or based on a detected ambient temperature, for example.
PMD 10 may be configured to determine the usable life or expiration time/date of the Product and notify the user of such information through a suitable output device 28.
PMD 10 may be configured to determine the health percentage of the product and output it to the user.
PMD 10 may output an alert (e.g., a visual or audible alert to a local user via an output device 28 and/or via wired or wireless interface(s) 30 to remote personnel) that indicates an estimated time before reaching a predicted temperature violation state.
PMD 10 may generate an alert signal to activate corresponding LED(s) or other output devices 28 to alert a user, which may allow a user to remedy the situation before reaching the temperature violation state, and thereby potentially preventing wastage or discarding of the Product.

The figures discussed below illustrate flowcharts of example algorithms 52 executable by processing unit(s) 22 of PMD 10, according to example embodiments. In some embodiments, PMD 10 may execute any or all of such algorithms.

FIG. 7 illustrates a flowchart of an example algorithm 130 for monitoring a Product using PMD 10 to determine an impending Product wastage condition, according to an example embodiment. At 132, PMD 10 monitors the temperature status of a Product, e.g., based on sensor data collected from one or more sensors 16. For example, PMD 10 may determine/calculate a current Product temperature, a predicted future Product temperature, and/or accumulated or historical Product temperature data. At 134, PMD 10 may determine the presence of an impending Product wastage condition based on sensor data collected and analyzed at 132. If an impending Product wastage condition is detected, PMD 10 may generate and output and/or transmit a notification at 136 indicating the impending Product wastage condition.

FIG. 8 illustrates a flowchart of an example algorithm 140 for monitoring a Product using PMD 10 to determine a temperature violation, according to an example embodiment. At 142, PMD 10 monitors the temperature status of a Product, e.g., based on sensor data collected from one or more sensors 16. At 144, PMD 10 may determine whether a determined temperature of the Product exceeds a relevant temperature threshold value (e.g., where the Product temperature is above a high temperature threshold value or below a low temperature threshold value), based on sensor data collected and analyzed at 142. If so PMD 10 may generate and output and/or transmit a notification at 146 indicating the temperature violation condition.

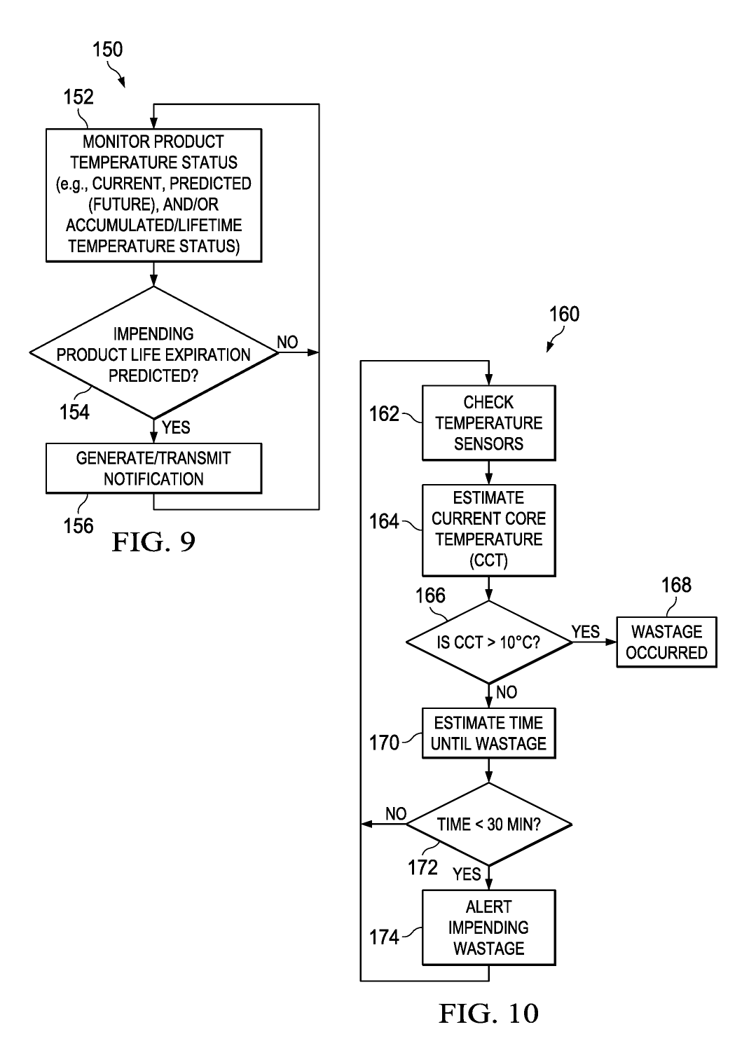

FIG. 9 illustrates a flowchart of an example algorithm 150 for monitoring a Product using PMD 10 to determine an impending Product life expiration condition, according to an example embodiment. At 152, PMD 10 monitors the temperature status of a Product, e.g., based on sensor data collected from one or more sensors 16. At 154, PMD 10 may determine the presence of an impending Product life expiration condition (e.g., the Product is approaching a predefined usable life time limit) based on sensor data collected and analyzed at 152. If an impending Product life expiration condition is detected, PMD 10 may generate and output and/or transmit a notification at 156 indicating the impending Product life expiration condition.

FIG. 10 illustrates a flowchart of an example algorithm 160 for monitoring a Product using PMD 10, according to an example embodiment. At 162, PMD 10 may receive temperature sensor data from one or more temperature sensors 18. At 164, PMD 10 may analyze the temperature sensor data to determine an estimated current core temperature (CCT) of the Product. At 166, PMD 10 may compare the determined CCT to a threshold temperature value, e.g., 10° C. If the CCT exceeds the threshold value, PMD 10 determines the presence of a Product wastage condition, at 168. Otherwise, the method proceeds to 170, at which PMD 10 determines an estimated time until Product wastage based at least on the temperature sensor data received at 162 (e.g., based on the determined CCT and a detected ambient temperature from an ambient temperature sensor 18C). At 172, PMD 10 may compare the determined estimated time until Product wastage to a threshold time period, e.g., 30 minutes. If the determined estimated time until Product wastage is less than the threshold time period, PMD 10 may generate and output/transmit an impending wastage alert at 174 (e.g., to a local user via an output device 28 and/or to remote personnel via wireless communication of an alert notification). Otherwise, the method may return to 162 to continue collecting temperature sensor data and monitoring the Product CCT.

Further, in some embodiments, PMD 10 may provide Product state feedback upon request by a user, e.g., via a button press or other user input via an input device 26. This information-on-demand system may save battery power. Alternatively, some embodiments stop warning users after the Product has passed a defined temperature threshold.

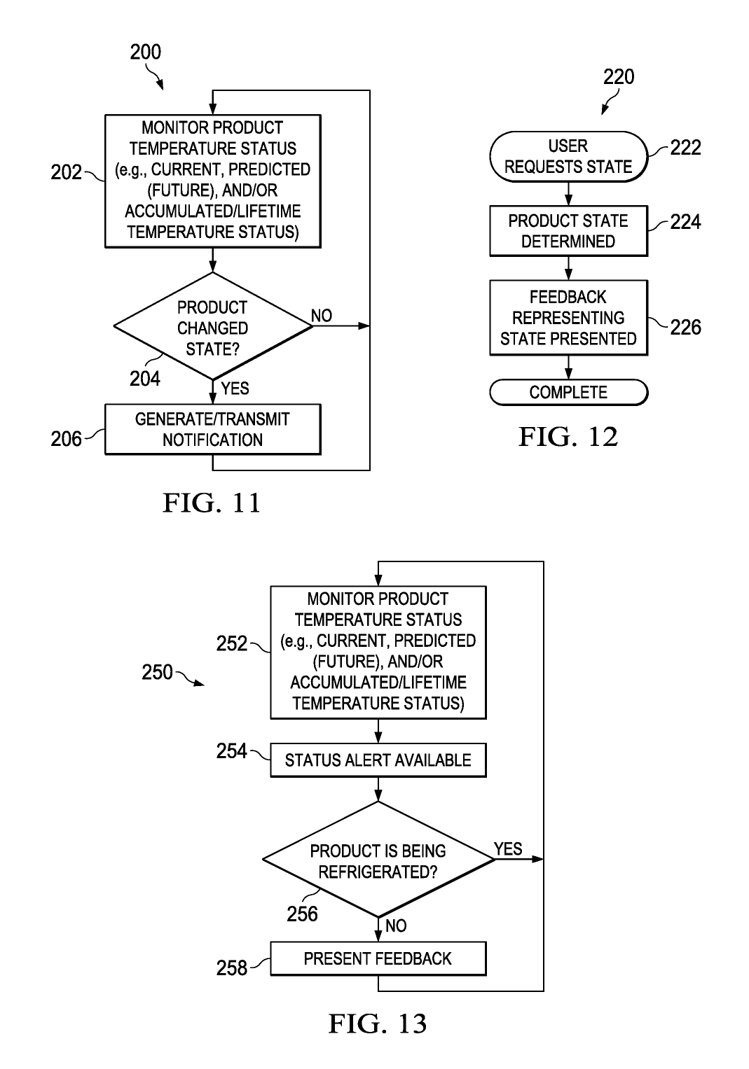

FIG. 11 illustrates a flowchart of an example algorithm 200 for monitoring a Product using PMD 10 to determine a state change of the Product, according to an example embodiment. At 202, PMD 10 monitors the temperature status of a Product, e.g., based on sensor data collected from one or more sensors 16. At 204, PMD 10 may determine a current state of the Product and identify a change in the Product state, based on sensor data collected and analyzed at 202. If a Product state change is determined at 204, PMD 10 may generate and output and/or transmit a notification at 206 indicating the Product state change and/or the current Product state, along with information related to the determined Product state or state change.

FIG. 12 illustrates an example algorithm 220 for outputting Product state information upon user request, according to an example embodiment. At 222, a user requests Product state information, e.g., by interfacing with a respective user input device 26, e.g., by pressing a button, by touching or making a gesture proximate a capacitive sensor, etc. At 224, PMD 10 may determine a current state of the Product, e.g., based on the most recent available sensor data or by initiating sensor readings in real time and evaluating such sensor data. At 226, PMD 10 may output the determined Product state to the user, e.g., via any suitable output device 28, e.g., an LCD display, one or more LEDs, via a speaker, via haptic feedback, or in any other manner.

In some embodiments, PMD 10 includes one or more accelerometers or other orientation sensor(s) 20C configured to detect orientation data or other data related to a physical orientation of PMD 10, and thus a physical orientation of the Product (e.g., assuming a known physical relationship between PMD 10 and the Product). PMD 10 may include suitable algorithm(s) 52 for analyzing the sensed accelerometer data and/or other orientation related sensor data to determine an orientation and/or movement of the PMD (and thus the Product), and/or to control aspects of the PMD operation based on the determined orientation and/or movement information. For example, PMD 10 may enable or disable selected output device(s) 28 based on the determined orientation and/or movement of the Product. As another example, PMD 10 may select a particular temperature calculation algorithm to apply (from multiple different temperature calculation algorithms corresponding to different Product orientations), or select or adjust one or more relevant threshold values for analyzing the state of the Product, based on the determined orientation and/or movement of the Product.

In some embodiments, PMD 10 may implement escalating alerts depending on the magnitude desired for a warning based on sensors 16 and stored values.

In some embodiments, PMD 10 is configured to enable alerts (communicated via output device(s) 28) only when the Product (e.g., blood product) is filled and in use. For example, PMD 10 may include ultrasonic, capacitive or other types of sensors 20 configured to detect the material of the Product, and/or optical sensors 20 configured to determine the color of the Product (e.g., blood bag) and detect to determine the extent to which the Product/Product packaging (e.g., blood bad) is filled.

Low Power Operation Techniques:

In some embodiments that are powered by a battery 32, PMD 10 may provide power-optimized operation to allow for longer battery life such that the PMD 10 may remain functional for the entirety of the relevant monitoring cycle or life expectancy of the Product.

In some embodiments, PMD 10 is a mobile battery-powered device that includes an ultra-low-power (ULP) MCU 22B with optimized sleep- and active-mode power consumption and power efficiency. Additionally, in some embodiments, the usage of the MCU 22B and the overall device 10 may be power optimized and efficient. For example, the MCU 22B may be controlled to stay in an active high-power consuming mode (e.g., about 1-10 mA) as little as effectively possible, perform its tasks quickly, and return to a low-power sleep mode (e.g., 100 nA-10 uA), in which the MCU 22B remains for a majority of the time. In one embodiment, the active time of the MCU 22B is about 10 ms for each 10 seconds, which defines an active duty cycle of about 1/1000. Maintaining PMD 10 in a state of deep sleep for significant periods may extend the battery life, e.g., for multiple years. Some embodiments of PMD 10 utilize FRAM, MRAM or similar non-volatile storage either external or built into MCU 22B, as such storage devices the energy efficiency, high speed, robustness and reliability of such storage devices, which makes it particularly suitable for battery powered and medical applications.

In some embodiments, PMD 10 may include ambient temperature sensor(s) 18C to improve the performance of the PMD, e.g., via improved battery life, based on the detected ambient temperature of the Product. For example, for a blood product that must be maintained at a low temperature, PMD 10 may execute an algorithm 52 to implement various PMD controls if the detected ambient temperature indicates that the blood product is located in a cooler/refrigerator, e.g., to effectively prevent determination of an alert state or disable generating or output of an alert notification (e.g., via an LED), and/or or remain in a lower power state or reduce a scheduled wake-up frequency if the ambient temperature indicates that the blood product is located in a cooler/refrigerator.

In some embodiments, the PMD processing unit(s) 22 may disconnect (power off) one or more temperature sensor(s) 18 (e.g., temperature sensor(s) 18 separate from the MCU 22B) when the PMD 10 is in an off state, e.g., to prevent power losses from leakage and thereby better optimize power consumption.

In some embodiments, temperature sensors 18 may help save power in one or more of the following manners.
For example, PMD 10 may vary the measurement frequency of sensor(s) 16 based on detected changes in the ambient temperature. For example, f PMD determines that the Product (e.g., blood bag) is in a cooler, PMD 10 may reduce the frequency of temperature measurements made by sensor(s) 18, and may later increase the temperature measurement frequency upon detecting an increase in ambient temperature (e.g., indicating that the Products has been removed from the cooler). Further, PMD 10 may be programmed to adjust the temperature measurement frequency as a function of a difference between the Product temperature and ambient temperature detected by sensors 18, e.g., PMD 10 may increase the measurement frequency as a function of increasing temperature difference between the detected Product temperature and detected ambient temperature.
As another example, to save power, PMD 10 may be configured to “deactivate” one or more thermistors 18 when they are not in use. PMD 10 may deactivate thermistors by effectively removing a ground connection in-between sequential measurements and allowing the thermistors to “float.” PMD 10 may perform such ground removal by connecting the low side of the thermistor network to the general-purpose input/output (GPIO) of MCU 22B and setting that GPIO to “high impedance” when measurements are not being taken. Alternatively, PMD 10 may turn off the ground by using an NMOS transistor connected between the low side of the thermistor network and the circuit ground. Activating the transistor allows current to pass through the thermistor, otherwise the transistor can be turned off and the thermistor network will “float.”
In some embodiments, when PMD 10 is idle, a PMD processing unit 22 may disconnect (power off) one or more components (e.g. temperature sensor(s) 18, data storage device(s) 24, output device(s) 28, user interface(s) 26, etc.), and/or configure the PMD processing unit GPIOs to a low power state with respect to the PMD circuit and therefore prevent power losses from current leakage.
As another example, PMD 10 may conserve battery life by providing only sensory feedback based on sensor data. For example, PMD 10 may use data from ambient temperature sensor(s) 18C to calculate whether the Product is in an environment where user feedback is useful or in an environment in which battery 32 can be saved, e.g., a cooler/refrigerator for blood products, e.g., according to the algorithm 52 shown in FIG. 13.

FIG. 13 illustrates an example algorithm 250 for controlling enabling/disabling the output of notifications via output device(s) 28, according to an example embodiment. At 252, PMD 10 monitors the temperature status of a Product (e.g., blood product), e.g., based on sensor data collected from one or more sensors 16. For example, PMD 10 may determine/calculate a current Product temperature, a predicted future Product temperature, and/or accumulated or historical Product temperature data. At 154, PMD 10 may determine, based on the sensor data collected and/or analyzed at 252, that an alert or notification condition regarding the Product is currently present. At 256, PMD 10 may further determine, based on the sensor data collected and/or analyzed at 252, whether the Product is currently located in a cooler (e.g., refrigerator), e.g., by comparing a detected ambient temperature (e.g., using an ambient temperature sensor 18C) with a threshold temperature value. If PMD 10 determines that the Product is currently located in a cooler, PMD 10 may disable or prevent the output of an alert or notification (corresponding with the condition determined at 254) via output device(s) 28. Alternatively, if PMD 10 determines that the Product is not currently located in a cooler, PMD 10 may output an alert or notification corresponding with the condition determined at 254 via output device(s) 28.

Sensor Data Acquisition and Processing

In some embodiments, PMD 10 may utilize one or more of the following tools or techniques to improve the accuracy of temperature measurements.

In some embodiments, PMD 10 include multiple sensors 16 of the same or different types and may use sensor aggregation algorithms. An algorithm may be used to combine multiple measurements into a single value to achieve higher accuracy and/or reliability through redundancy and heterogeneous data. In one embodiment, a Kalman filter or similar filter, may assign varying “weights” to different sensors 16 and their time-series measurements. For example, a temperature of the Product can be calculated based on a weighted average of (a) a temperature measurement of an MCU internal on-chip temperature sensor 18D of the MCU 22B and (b) a temperature measurement of a product-interfacing temperature sensor 18A, e.g., in direct thermal contact with the Product, wherein the measurement of the product-interfacing temperature sensor 18A is assigned a first weight A (e.g., 0.85) and the measurement of the MCU internal on-chip temperature sensor 18D is assigned a second, different weight B (e.g., 0.15).

In some embodiments, PMD 10 may utilize linearization. For sensors 16 with complex transfer characteristics, linearization techniques may be utilized to improve measurement accuracy and/or reduce the computational load of PMD 10. Linearization may be implemented by software or hardware included in PMD 10. For example, thermistors change resistance exponentially, ADC and computation error can accumulate quickly, especially in low resolution or low voltage systems. Thus, PMD 10 may utilize a linearization function to modify the transfer characteristics of the sensor and “linearize” its output through use of software or by means of a special hardware conditioning circuit (e.g., any or a combination of one or multiple passive elements or active elements such as a resistor(s), capacitor(s), inductor(s), operational amplifier(s), transistor(s), etc.). In some embodiments the full transfer characteristic of a sensor may not be ideally linearized, but rather linearization within required error may be achieved in a specific region of the sensor operation. An example solution is to pick a region of interest (e.g., 40° C.+/−20° C.) around the focal point or the “ideal” temperature for the respective Product, and by adding a single resistor in parallel with the thermistor, effectively make the output close to linear for that temperature range. This may limit the absolute range of the thermistor, but allow PMD 10 to determine accurate measurements while using very little processing power when in the defined temperature region.

In some embodiments, PMD 10 may utilize an adjustable linearization. Some embodiments of PMD 10 can adjust or “tune” the region in which a thermistor output is linear by using digital potentiometers or by switching passive components in and out using relays or transistor-based solid state switches.

In some embodiments, PMD 10 may use a product-interfacing temperature sensor 18A to monitor the Product as long as the product-interfacing temperature sensor 18A is working properly, but automatically switch over to using an MCU internal on-chip temperature sensor 18D as a backup temperature sensor when the product-interfacing temperature sensor 18A is malfunctioning or otherwise not working properly, as determined by PMD 10.

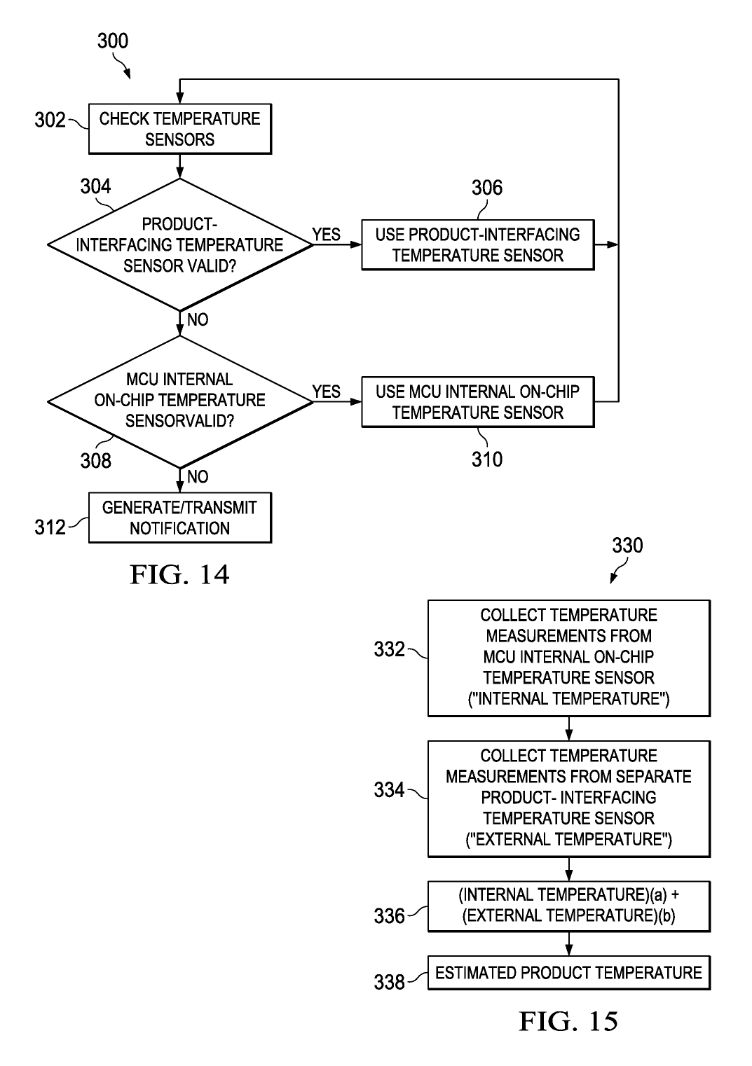

FIG. 14 illustrates an example algorithm 300 implemented by PMD 10 for providing such redundant temperature monitoring, according to certain embodiments. In such embodiments, PMD 10 may include a product-interfacing temperature sensor 18A, an MCU internal on-chip temperature sensor 18D, and/or additional temperature sensor(s) 18 and/or other sensor(s) 20. At 302, PMD 10 may receive measurements from at least the product-interfacing temperature sensor 18A and MCU internal on-chip temperature sensor 18D, e.g., at a defined measurement frequency.

At 304, PMD 10 may determine whether product-interfacing temperature sensor 18A is valid (e.g., operating properly) using any suitable evaluation process. For example, PMD 10 may determine that sensor 18A is “invalid” if sensor 18A fails to provide measurement data to a relevant processing unit 22 for a specified time period (e.g., a defined number of measurement cycles). As another example, where sensor 18A is providing measurement data, PMD 10 may compare the measurement data received from sensor 18A with data received from other temperature sensor(s) 18 and/or other sensor(s) 20 of PMD 10, to determine whether the measurement data received from sensor 18A is inconsistent with the other sensor data (e.g., by determining whether data from sensor 18A differs from data from other sensor(s) 18 by more than a defined threshold amount). As another example, PMD 10 may compare current or recent measurements from sensor 18A with previous measurements from sensor 18A to detect an abnormal trend or change in measurement data from sensor 18A, such as a sudden temperature spike (high or low) or other abnormal temperature trend, e.g., by comparing data from sensor 18A with defined thresholds or defined ranges regarding “normal” or “valid” temperature measurements, temperature trends, temperature changes, etc. In some embodiments, PMD 10 may analyze a first or second derivative of measurement data from sensor 18A, e.g., to determine an abnormal rate of temperature change (e.g., as defined by respective threshold values).

If PMD 10 determines that the product-interfacing sensor 18A is valid (e.g., functioning properly), PMD 10 may use the measurement data from sensor 18A (by itself of in combination with other sensor data) for analyzing a current or future Product temperature or Product state, as indicated at 306. Alternatively, if PMD 10 determines that the product-interfacing sensor 18A is not valid (e.g., not functioning properly), PMD 10 may then check whether MCU internal on-chip temperature sensor 18D may be used (at least temporarily) in place of the product-interfacing sensor 18A for analyzing a current or future Product temperature or Product state. Thus, at 308, PMD 10 may determine whether MCU internal on-chip temperature sensor 18D is valid (e.g., operating properly) using any suitable evaluation process, e.g., any process discussed above regarding the validation of sensor 18A at 304. If PMD 10 determines that the MCU internal on-chip temperature sensor 18D is valid (e.g., functioning properly), PMD 10 may being using the measurement data from sensor 18D in place of the product-interfacing sensor 18A for analyzing a current or future Product temperature or Product state, as indicated at 310. (In some embodiments, PMD 10 may later switch back to sensor 18A if PMD 10 determines that sensor 18A begins functioning properly again.) Alternatively, if PMD 10 determines at 308 that the MCU internal on-chip temperature sensor 18D is invalid (e.g., not functioning properly), i.e., where neither sensor 18A nor 18D is functioning properly, PMD 10 may generate and output/transmit an alert notification at 312 indicating this condition. Alternatively, in an embodiment in which PMD 10 includes additional temperature sensor(s) 18, PMD 10 may switch over to one of such additional temperature sensors 18.

In some embodiments, PMD 10 uses data from both an MCU internal on-chip temperature sensor 18D and a separate product-interfacing temperature sensor 18A to determine the Product temperature.

FIG. 15 illustrates an example algorithm 330 for calculating a Product temperature as a weighted average of the temperature readings of an MCU internal on-chip temperature sensor 18D and a separate product-interfacing temperature sensor 18A, according to an example embodiment. At 332 and 334, PMD 10 may collect (a) temperature measurements from MCU internal on-chip temperature sensor 18D, which measurements may be referred to as “Internal Temp” measurements, and (b) temperature measurements from product-interfacing temperature sensor 18A, which measurements may be referred to as “External Temp” measurements. At 336, a PMD processing unit 22 may combine the “Internal Temp” and “External Temp” measurements using any mathematical equation. For example, as shown at 336, the processing unit 22 may applying different weights (e.g., constants or multipliers “a” and “b”) to the “Internal Temp” and “External Temp” measurements, respectively, and adding the results to output an estimate Product temperature at 338.

State Determination/Decision Making

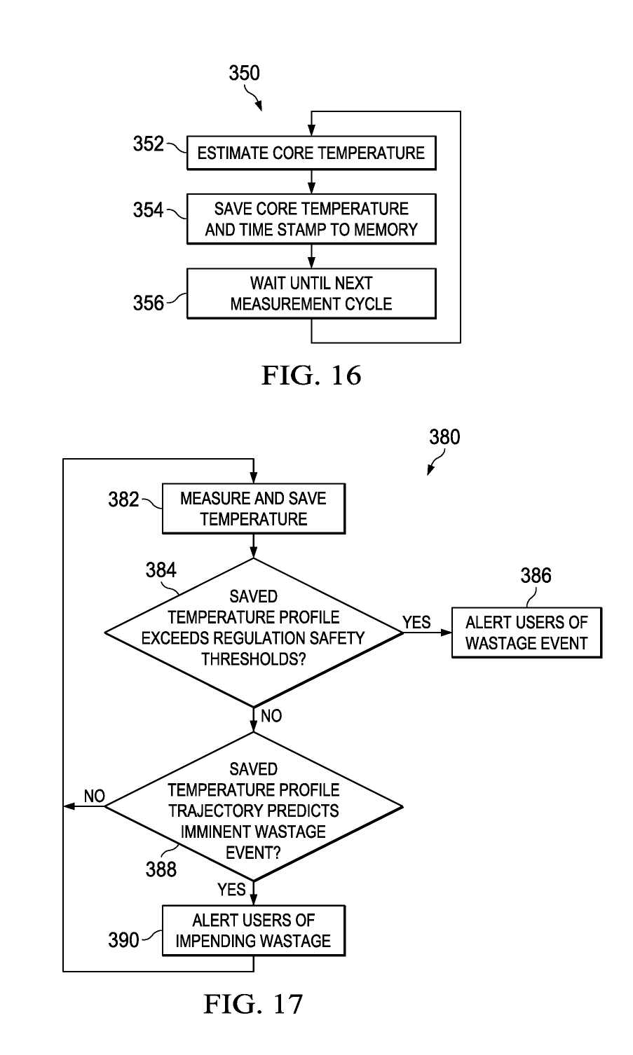

FIG. 16 illustrates an example algorithm 350 executed by PMD 10 to monitor a Product (e.g., to monitor the temperature of a blood product), according to some embodiments. At 352, PMD 10 may determine or calculate a core temperature of the Product, e.g., using any algorithms or techniques disclosed herein. At 354, PMD 10 may store the core temperature and time stamp in memory/data storage 24, wait until the next measurement cycle at 356 (e.g., according to a defined measurement frequency or schedule), and then repeat the process.

In some embodiments, PMD 10 may run different software configurations on the same hardware configuration. For example, in case of various types of blood products (e.g., PRBC, FFP, platelet, cryo, etc.), different parameters may be monitored or different rules have to be followed to determine the state of the Product. In such cases, PMD 10 may be programmed at the time of manufacturing or configured directly in the field to comply with the requirements for the specific Product being monitored. This may allow PMD 10 to be dynamically configured for use with a wide range of different Products. PMD 10 may be configured directly through any available physical interface or wirelessly using any wireless technology supported by PMD 10.

FIG. 17 illustrates an example algorithm 380 executed by PMD 10 to monitor a Product (e.g., to monitor the temperature of a blood product) to detect an occurred or impending Product wastage event, according to one embodiment. At 382, PMD 10 measures Product temperature data using one or more temperature sensors 18 and saves such temperature data in memory 24. At 384, PMD determines whether the saved Product temperature data exceeds a defined threshold value corresponding with a current Product wastage event, e.g., defined by industry-relevant regulations for the Product. If so, at 386, PMD 10 may output and/or transmit an alert or notification of the current Product wastage event.

If PMD 10 determines at 384 that the saved Product temperature data does not currently exceed the defined threshold value corresponding with a current Product wastage event, the method advances to 388, where PMD 10 determines whether a Product wastage event (which has not yet occurred) is imminent, based on the Product temperature data measured and saved at 382. For example, PMD 10 may (a) determine a predicted future Product wastage event based on the saved Product temperature data (e.g., based on a temperature trend that exceeds a threshold value at some point in the future), and (b) determine whether the predicted future Product wastage event is “imminent,” e.g., by (i) calculating a projected time from the current time until the predicted future Product wastage event, e.g., based on a calculated trend (e.g., rate of change) of the saved Product temperature data, and (ii) determine whether the projected time for the predicted future Product wastage event is within a defined “imminent” time threshold (e.g., 30 minutes).

If PMD 10 determines at 388 the presence of an imminent Product wastage event, PMD 10 may output and/or transmit an alert or notification of the imminent Product wastage event, at 390. If not, the method may return to 382 to continue monitoring for existing or imminent Product wastage events.

In some embodiments, PMD 10 may execute complex algorithms 52 to analyze the state of a Product, e.g., to identify a current and/or predicted state from a group of possible states defined by PMD 10. One or more defined states for a Product may correspond with a regulation concerning that Product. For example, for blood products, one or more states may be defined by a hospital or regulatory body. For instance, assuming a regulation that blood must be stored between 1° C. and 10° C., PMD 10 may define an “acceptable temperature” state indicating that the blood product is between 1-10° C., a “low temperature violation” status indicating that the blood product is below 1° C., and a “high temperature violation” state indicating that the blood product is above 10° C. In this example, the low temperature violation state and high temperature violation may be referred to as “wastage states” of a blood product, e.g., where the regulations require that the blood product be discarded after such temperature violations.

The relevant regulations (for example, temperature thresholds, allowable durations at particular temperatures, etc.) may change over time, and the relevant PMD algorithms 52 may be dynamically adjusted accordingly. Currently, for RBC and FFP blood products, the AABB mandates 1-6° C. for storage and 1-10° C. for transport. Products that are above the 10° range when checked back into a blood bank are considered wasted. PMDs 10 can be customized to fit whatever wastage logic is in place at a hospital.

In some embodiments, PMD may use different algorithms 52 based on type of blood product, size of blood product and other physical characteristics e.g., adult vs. pediatric unit.

Some types of Products only enter a wastage stage after existing in a temperature violation state for some defined period of time. Thus, for such Product types, PMD 10 may keep track of the time-based temperature profile of the respective Product, in order to determine not only temperature violation, but also whether a wastage state has been reached. The same may similarly apply to Product Characteristics other than temperature, depending on the particular type of product and embodiment or configuration of PMD 10.

Unlike chemical indicators, PMD 10 may be configured to determine the state of a Product using arbitrary modifiable logic and updatable software 52.

As discussed above, PMD 10 may be configured to determine not only a current state of a Product, but also a predicted future state of the Product, e.g., by determining and analyzing projected temperature trajectories for the Product. Thus, PMD 10 can detect a temperature trajectory approaching a violation or wastage state and thus generate an alert signal to output an alert notification. PMD 10 can also detect a product temperature that is stable but close to a violation threshold. PMD 10 may assign a different status to such condition (i.e., as distinguished from a temperature trajectory approaching a violation or wastage state), and may or may not output a corresponding alert signal, depending on the embodiment or device setting. Further, a Product is neither close to nor headed towards a violation or wastage state may be classified as good and stable.

In some embodiments, Product (e.g., blood product) violation or wastage state thresholds may be dynamic. PMD 10 may trigger alerts based on amount of time within and outside of a defined temperature range, based on a magnitude of temperature deviation, based on the product life cycle, or based on any other relevant parameters or events. Violation or wastage can be determined based on a combination of duration of temperature violation, magnitude of temperature violation, age of blood product, etc. For example, consider that an older product may waste quicker than a fresher product. Because PMD 10 may include complex algorithms 52 programmed into the MCU 22B, PMD 10 need not analyze the status of the Product in a linear manner such as with chemical indicators; rather, PMD 10 may use different rules for different sets of sensor inputs and time parameters.

In some embodiments, PMD 10 may be programmed to algorithmically ignore transient and abrupt temperature deviations, and/or to apply smoothing, averaging, or other data filtering or conditioning formulas.

In some embodiments, PMD 10 may be programmed to deal with special cases such as variance due to condensation or physical movement.

In some embodiments, PMD 10 may apply thermodynamics equations to calculate a current temperature of the Product core, as well as a temperature trajectory for calculating a predicted future temperature. PMD 10 may utilize ambient temperature measurements to facilitate or improve the accuracy of such calculations.

Further, PMD 10 may also use ambient temperature measurements as input for determining whether to ignore particular abrupt changes in the detected Product temperature (e.g., deviations in a surface temperature of the Product).

In some embodiments, PMD 10 may be configured to determine and maintain temperature history or and/or logs 64 including a record of lifetime heating of a Product (e.g., blood product) using particular software algorithms 52, e.g., for a low memory configuration, which may include one or more of the following:
Exposure to excessive temperatures accumulated over time: in some embodiments, PMD 10 can keep track of total time that a Product (e.g., blood bag) has been above a certain temperature, as well as detecting and tracking levels of exposure as shown in example below. This may be performed using a relatively small amount of memory space in memory/storage device(s) 24 of PMD 10, and may provide accurate results. An arbitrary example is as follows:
   a. Level 1>2° C. max allowed time 10 hours
   b. Level 2>5° C. max allowed time 5 hours
   c. Level 3>10° C. max allowed time 1 hour
   d. Level 4>15° C. max allowed time 20 min
   e. Level 5>20° C. max allowed time 1 min
Averaging/Filtering: in some embodiments, PMD 10 may utilize simple techniques such as using buffers with multiple data bins, FIR/IIR filters or an exponential moving average formula to effectively smooth out the average temperature changes over a certain period of time.
Storing individual samples: in some embodiments, PMD 10 may include an external memory chip 24, e.g., a flash memory chip, that stores all sensor data collected by the PMD sensor(s) 16 and processes such data to determine very accurate details of the temperature profile/changes of the Product.
In some embodiments, PMD 10 extends the mimicry example discussed above, and instead of the predefined levels, executes a more complex algorithm 52 to calculate the average exposure level and an average exposure time. Such algorithm 52 may include multiplying the exposure level by the exposure time to determine a threshold value, wherein the limit of the product life is defined by such threshold value.

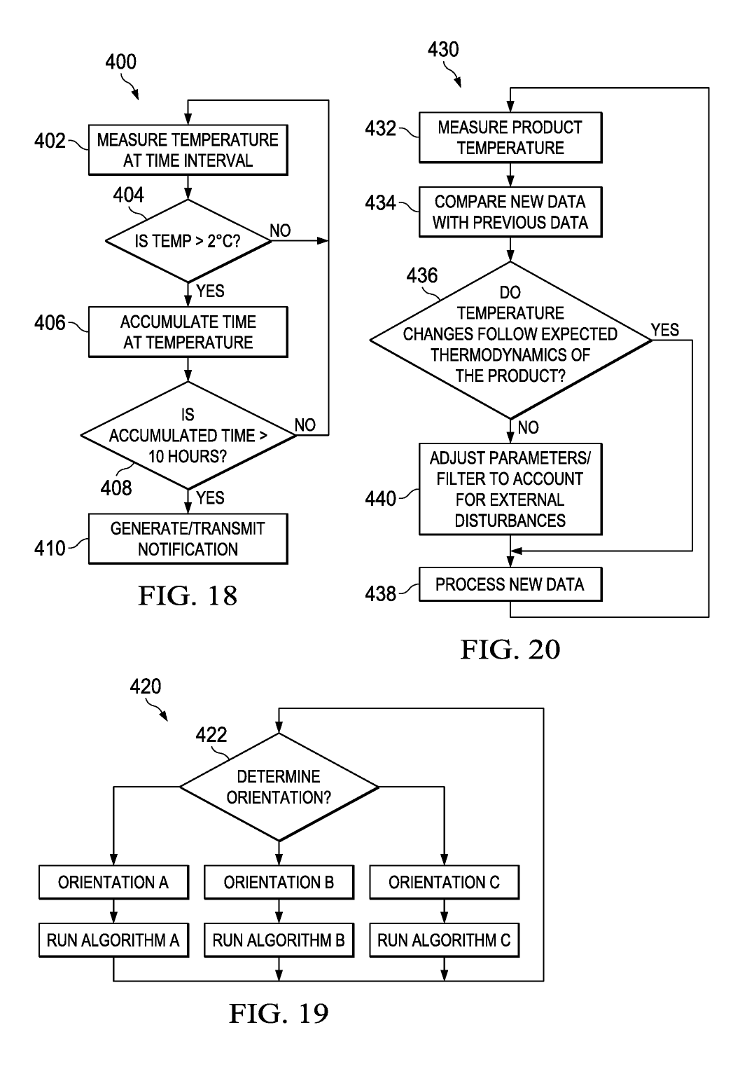

FIG. 18 illustrates an example algorithm 400 executable by PMD 10 to monitor accumulated or “lifetime” heating of Product, according to an example embodiment. At 402, PMD 10 determines a Product temperature based on measurements from one or more temperature sensor(s) 18, at a defined measurement frequency or interval. At 404, PMD 10 determines whether the determined Product temperature exceeds a defined threshold, e.g., above 2° C. If so, PMD 10 starts or increases an accumulated time counter for recording an accumulated time in which the Product exceeds the threshold temperature (e.g., above 2° C.). For example, if the temperature is measured at 5 second intervals, for each determination at 404 of the Product temperature exceeding the defined threshold value, PMD 10 may increase the accumulated time counter by 5 seconds. At 408, PMD may determine whether the accumulated time counter exceeds a predefined duration, e.g., 10 hours. If so, at 410, PMD 10 may output and/or transmit an alert or notification regarding the accumulated time (e.g., above 10 hours) exceeding the defined threshold temperature (e.g., above 2° C.).

In some embodiments, PMD 10 may include an accelerometer, tilt switch, or other orientation sensor(s) 20C to determine the position and/or orientation of the Product. This capability may be particularly useful in situations in which the current orientation of the Product may require a change in the algorithm 52 used by PMD 10 to estimate the core temperature of the Product. For example, a blood product may experience different rates of heating/cooling when influenced only by thermal convection (e.g., when the blood product is hanging) versus when influenced by a combination of thermal convection plus conduction (e.g., when the blood product is lying on a table). In such cases two or more different algorithms 52 for calculating the Product core temperature based on data received from temperature sensor(s) 18 can be programmed in the PMD and be selectively implemented by PMD 10 based on the detected orientation of the bag (as determined by orientation sensor(s) 20C) to provide an accurate core estimation.

FIG. 19 illustrates an example algorithm 420 executed by PMD 10 for selectively implementing different core temperature calculation algorithms 52 based on a Product orientation determined using orientation sensor(s) 20C, according to an example embodiment. At 422, PMD 10 may determine an orientation of the Product (e.g., from a number of predefined Product orientations) or may determine orientation parameters of the Product (e.g., one or more angles or inclination in one or more planes or along one or more axes). In the illustrated example, PMD 10 may determine whether Product is current in one of three different predefined orientations: Orientation A (e.g., hanging), Orientation B (lying on a first side of the Product packaging), or Orientation C (lying on a second side of the Product packaging). PMD 10 may then select and implement a respective core temperature determination algorithm 52 based on the determined Product orientation. In this example, PMD 10 implements core temperature determination Algorithm A while the Product is in Orientation A, implements core temperature determination Algorithm B while the Product is in Orientation B, and implements core temperature determination Algorithm C while the Product is in Orientation C. PMD 10 may switch between Algorithms A, B, and C as the Product orientation switches between Orientations A, B, and C over time.

In some embodiments, PMD 10 may run algorithms that follow special rules or exceptions. For example, PMD 10 may judge viability of the Product as a factor of both time and temperature. Because the guidelines in place may allow the Product to exceed or stay within specific temperature ranges for a limited amount of time, PMD 10 may be configured to strictly follow these guidelines and use a “soft” spoilage threshold where not only critical core temperature triggers a spoilage event but rather a combination of specific temperatures maintained or exceeded for specific time would trigger such an event. Due to flexible nature of the design of the PMD software/algorithms 52, PMD 10 may be able to strictly follow complicated regulations and guidelines and provide “close to spoilage” alerts as well as detect Product spoilage based on many combined factors. In some embodiments, GPS data or accelerometer data may be used to determine if the Product is being transported. If active transport state is detected, PMD 10 may switch to an alternative algorithm 52 to meet the transport monitoring guidelines for the Product if they happen to be different from storage monitoring guidelines.

False Positive/Negative Detection

In some embodiments, PMD 10 may prevent false positive or false negative alerts from PMD 10 by distinguishing between expected gradual changes of a monitored Product Characteristic due to various environmental factors and brief transient changes, e.g., due to temporary handling of the Product. For example, PMD 10 may prevent a false positive alert caused by a warm hand touch of a Product by filtering out rapid ambient temperature transients and adjusting relevant temperature calculations while the transient event is occurring. After the passing of the transient event, PMD 10 may return to normal operation and thus potentially avoid erroneously determining a Product wastage event.

Increased Accuracy Through Redundancy

Some embodiments of PMD 10 may use multiple temperature sensors 18 to capture one or more spatial temperature gradients with higher accuracy and reliability. PMD 10 may also provide improved environment sensing, for example, by sensing temperature from multiple sides of the Product, at multiple locations on the Product, and/or at multiple distances from the Product. For example, PMA 10 may be configured to filter out transient local disturbances from the environment or nearby personnel by sensing such transient local disturbances at one or more sensors but not at one or more other sensors. This concept may also be incorporated in a core temperate calculation algorithm executed by PMD 10, e.g., where direct measurement of the core temperature is not available. For example, some embodiments may use surface sensors on multiple sides of the Product to improve the analysis of the relevant heat transfer, e.g., if the Product is exposed to different environments, for example, exposed to ambient air on one side, but in physical contact with a solid or liquid surface on the other that will affect the nature of heat transfer through the surface area of such physical contact. Some embodiments of PMD 10 may use multiple sensors 18 at different distances from the Product to allow for a more complex model more accurately capturing the spatial gradient and as a result more accurately deriving other Product characteristics. e.g. core temperature. Some embodiments may use multiple sensors 18 inserted inside the Product to precisely measure the spatial temperature distribution and dynamics of the Product.

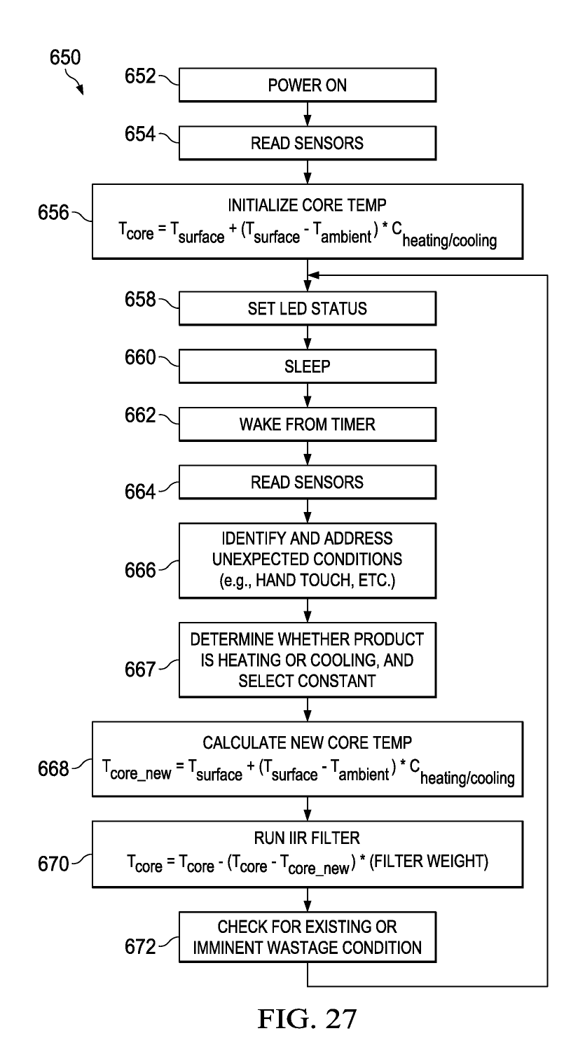

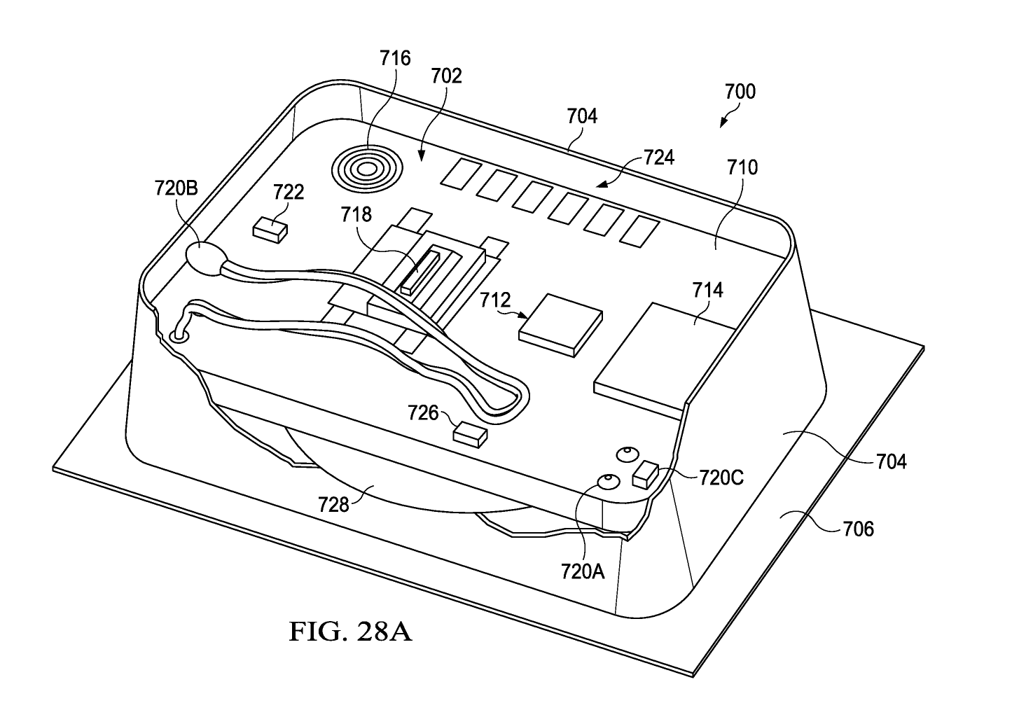

FIG. 20 illustrates an example algorithm 430 executable by a PMD processing unit 22 to analyze the validity of sensor data generated by PMD sensor(s) 18. At 432, processing unit 22 may measure or determine a Product temperature. This may entail simply taking readings from one or more temperature sensors 18, or calculating a Product temperature (e.g., Product core temperature) using any technique or algorithm disclosed herein (e.g., algorithm 650 discussed below with reference to FIG. 27).

At 434, processing unit 22 may compare the Product temperature measured or calculated at 432 with one or more previous Product temperature measurements or calculations, and determine a temperature change. At 436, processing unit 22 may determine whether a temperature change determined at 434 follows expected thermodynamic laws or parameters for the Product being monitored, e.g., by comparing the temperature change to one or more threshold values, or by performing a more complex analysis of the temperature change data over time.

If it is determined that the monitored temperature is following expected thermodynamic laws or parameters, the method proceeds to 438 and then 432 to process new data. However, if it is determined at 436 that the monitored temperature violates an expected thermodynamic law or parameter, PMD 10 may dynamically adjust its operation to account for such exception(s). For example, during a detected hand touch event, ambient sensor(s) 18C of PMD 10 may experience rapid heating. During this phase, PMD 10 may adjust thermal data filters used in the temperature calculation algorithm (e.g., this may involve decreasing a filter weight for subsequently processed data, which may result in a lesser impact on current data) such that the event may have a reduced or slight impact on the estimated Product core temperature calculated by PMD 10. After PMD 10 determines that the transient temperature has subsided, PMD 10 may return to normal operation. Another example for unexpected behavior includes detection of thermal readings that are too high or too low with respect to normal operating temperatures for the respective type of Product, according to predefined threshold value(s) for temperature (e.g. below −50° C. or above +100° C.), temperature change, or rate of change, for example. In this case, PMD 10 may ignore the data determined to be erroneous and rely on remaining sensor(s) 18 to analyze the thermal situation. If the readings of the erroneous sensor(s) 18 return back to normal after some time, PMD 10 may resume normal operation, including sensor data from such sensor(s).

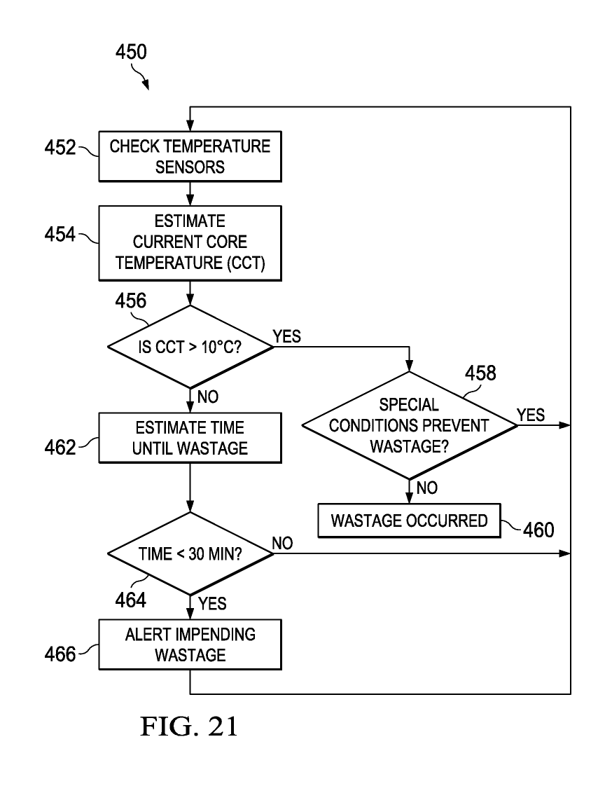

FIG. 21 illustrates an example algorithm 450 executed by PMD 10 for monitoring a Product to identify a current or imminent Product wastage event, according to an example embodiment. At 452, PMD 10 may receive measurements from one or more temperature sensors 18. At 454, PMD 10 may determine or calculate a current core temperature (CCT) of the Product, e.g., using any technique or algorithm disclosed herein. At 456, PMD 10 may determine whether the Product CCT exceeds a defined threshold value (e.g., above 10° C.). If the Product CCT does exceed the defined threshold value, at 458, PMD 10 may determine the presence (or not) of any defined special condition that would prevent Product wastage, e.g., regional regulations requiring the threshold to be exceeded for a certain period of time, false positive/negative prevention algorithm determining that the wastage event is actually a transient and false condition, etc. If such a special condition does exist, PMD 10 determines there is no Product wastage condition and the method returns to 452 for continued temperature monitoring. Alternatively, if it is determined at 458 that no special condition exists that would prevent Product wastage, PMD 10 determines a current Product wastage condition at 460 and may output/transmit a relevant Product wastage notification.

Returning to step 456, if the Product CCT does not exceed the defined threshold value, at 462, PMD 10 may determine an estimated time until Product wastage, based on stored temperature data collected over a number of temperature measurement intervals at 452. In some embodiments, determining an estimated time until Product wastage may involve calculating a future time at which the Product will exceed the defined threshold value (e.g., above 10° C.) corresponding with a Product wastage condition. In other embodiments, determining an estimated time until Product wastage may involve calculating a future time at which the Product will have spent an defined accumulated amount of time (e.g., above 5 hours) at a temperature exceeding a different defined threshold value (e.g., above 5° C.), wherein such time and temperate parameters may be defined by relevant regulations for the respective type of Product. In some situations, PMD 10 may determine that no future Product wastage is predicted, based on the current state of the Product (e.g., where the Product temperature is within a defined temperature range and stable. At 464, PMD 10 may determine whether the estimated time until Product wastage (if any) determined at 462 within a defined time period, e.g., less than 30 minutes from the current time, and if so, PMD may identify an imminent Product wastage condition and output/transmit an alert indicating the imminent Product wastage, at 466.

Core Temperature Calculation

In some embodiments of PMD 10, e.g., embodiments that do not directly measure the core temperature of the Product (e.g., embodiments that do not include any internal product sensor 18B), PMD 10 may derive the Product core temperature from other correlated characteristics measured by sensor(s) 16 or derived from sensor data, e.g., ambient temperature dynamics, surface temperature dynamics, spatial temperature distribution, etc. Some embodiments of PMD 10 may utilize a generalized heat equation or simplified heat transfer models, for example, a lumped static resistive model, newton's equation, etc. Some embodiments may use a combination of models to improve reliability, robustness, and/or accuracy of the core temperature derivation algorithm In some embodiments, PMD 10 may use a time-correlated dynamic algorithm for calculating an estimated Product core temperature. In general, PMD 10 may use a single or multiple temperature sensor(s) 18 to take carefully timed measurements and correlate them with a target parameter over time. Temperature dynamics derived from the external temperature sensors 18 may be correlated with the Product core temperature by accounting for the thermal mass of the Product being monitored. For example, if PMD 10 is aware of the mass of a blood product, the time intervals of the temperature measurements from sensor(s) 18, and the changes in temperature measurements during that time, then PMD 10 can determine external environmental factors influencing the blood product as well. Based on the above, PMD 10 can determine a simple thermal resistance model, or a more complicated thermodynamic model of the heat transfer to or from the blood product core. PMD 10 can then reliably calculate an estimated core temperature using the established core temperature calculation model.

Alternatively, PMD 10 may utilize the fact that the Product core temperature is bound by (if the heat source/sink is external/outside) the surface temperature (e.g., during heating the Product core is lower or equal the Product surface, and during cooling the core is hotter or equal to surface) and can be approximated using a simple offset. PMD 10 may adjust proportionally to a determined rate of the temperature change at the Product surface.

In another embodiment, the core temperature estimation algorithm implemented by PMD 10 may model the heat transfer process (e.g., Newton's heat equation) to estimate the core temperature of the Product. PMD 10 may directly measure or estimate the temperature of the heat source/sink (environment). The value of the estimated Product core temperature may be determined as the previous estimated Product core temperature plus a determined temperature change that occurred during a time period since the most recent estimate. This temperature change due to heat transfer is correlated with the temperature difference between the core temperature of the Product and the environment external to the Product, as well as the time period during which the heat transfer is being estimated. In this way, PMD 10 can estimate the Product core temperature based on a simplified heat transfer equation. Alternatively, the core temperature estimation algorithm may use a low pass filter to model the Product's thermal behavior, e.g., the finite mass and heat transfer rate of the Product. Assuming the heat source/sink is external to the Product, the core temperature of the Product will follow the heat source/sink with a certain time delay proportional to the mass of the Product. Therefore, the weight of the low pass filter may be selected to accurately represent the thermal mass of the Product.

PMD 10 may use one or a combination of multiple algorithms to calculate the estimated Product core temperature. A combination of multiple algorithms may be used to improve the adequacy of the model under different environments and to provide redundancy, parameter correction, error sanity checking, and/or improved accuracy.

Handling Variations

Some embodiments may use one or a combination of the models to improve reliability, robustness and accuracy of the core derivation algorithm. A combination of multiple algorithms 52 may be used to improve the adequacy of the model accounting for product parameters and environment factors variations and to provide redundancy, parameter correction, error sanity checking, better accuracy. Important product parameter variations that a model may need to account for include: shape, orientation, volume, mass, material type/heat capacity, thermal characteristics of the packaging, etc. Important environmental factors may include, for example: type of heat transfer and contribution of each (e.g. convection, conduction, radiation, normal or forced airflow in convection, exposed surface to each type of heat transfer, transient disturbances from the environment or personnel, etc.).

Two typical examples of such variations is exposure to different heat transfer types depending on the orientation and position of the Product. The Product may be suspended hanging in the air exposed to convection through the ambient air surrounding from all sides or the product may be laying on a surface with only one side exposed to ambient air convection and with other side in contact with a liquid or solid surface blocking it from the ambient air convection but facilitating thermal conduction through the surface area in contact. Depending on these factors, the core temperature dynamics of Product may be different.

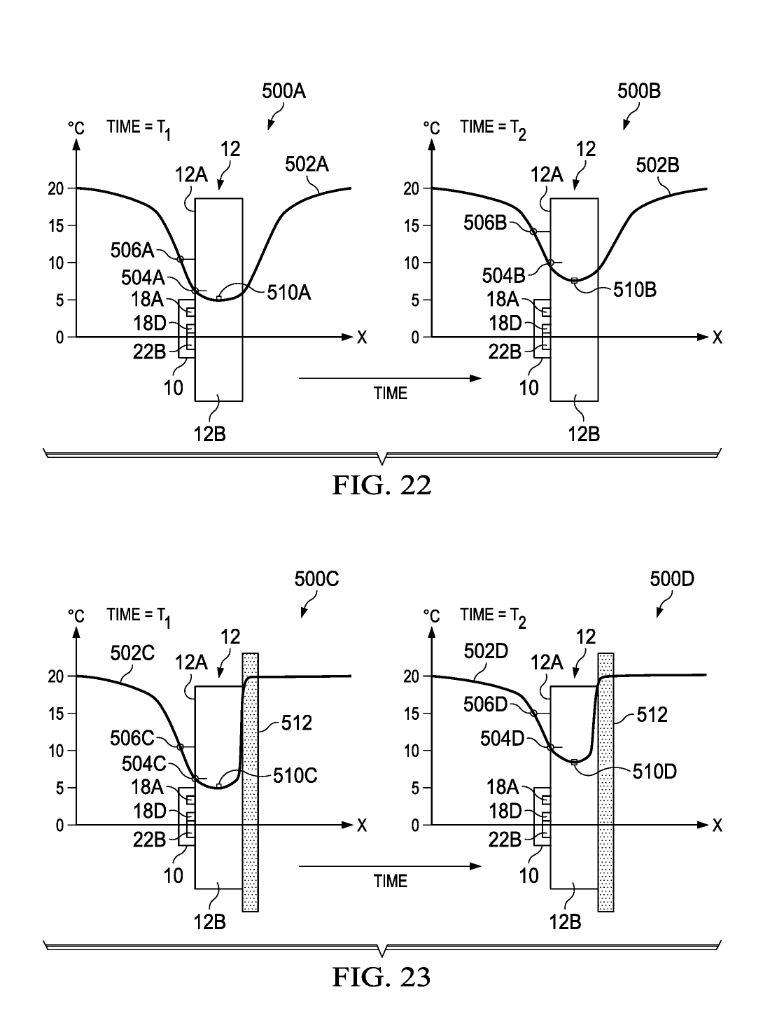

FIG. 22 illustrates an example temperature curve extending from ambient (on two opposite sides of the Product) and through the Product core, in a situation in which the Product is thermally influenced only (or substantially only) by convection, i.e., via transfer of heat by the movement of fluids (including air molecules and Product molecules). For example, FIG. 22 may represent a situation in which the Product is hanging in open air. FIG. 22 shows a first graph 500A showing a temperature curve 502A relative to the Product at a first time T₁, and a second graph 500B showing a temperature curve 502B relative to the Product at a second T₂ subsequent to first time T₁, which pair of graphs illustrates an upward shift in the temperature curve over time from T₁ to T₂, toward the ambient temperature. FIG. 22 may represent a situation in which a cooled Product is suddenly exposed to a higher ambient temperature (e.g., when the Product hung in a cooler is removed from a cooler and hung in an ambient room), wherein graph 500A indicates the temperature situation shortly after the cooled Product is exposed to the higher ambient temperature, and 500A indicates the temperature after more time exposed to the higher ambient temperature.

Graph 500A shows a cross-section of an example Product 12, e.g., blood product, including a Product packaging 12A (e.g., blood bag) that contains a temperature-sensitive fluid 12B (e.g., blood), and a PMD 10 secured to the Product packaging 12A. PMD 10 may include a product-interfacing temperature sensor 18A arranged to measure a surface temperature of the Product 12, a microcontroller 22B including an MCU internal on-chip temperature sensor 18D, and/or one or more other sensors 16. Temperature curve 502A extends from an ambient temperature on opposing sides of Product 12, in this example a 20° C. ambient temperature, through the core of the Product, in this example a core temperature of 5° C. indicated at 510A. Thus, as Product 12 is exposed to an ambient temperature higher than the Product core temperature, the Product core temperature will increase over time, toward the ambient temperature. As shown, temperature curve 502A is symmetrical about the Product core, assuming the same heat transfer influences on each side of the Product 12.

Graph 500A also shows temperature values 504A and 506A along temperature curve 502A measured by product-interfacing temperature sensor 18A and MCU internal on-chip temperature sensor 18D, respectively. In the illustrated example, temperature sensor 18A measures a value 504A of approx. 6° C., and temperature sensor 18D measures a value 506A of approx. 10.5° C. PMD 10 may execute any suitable core temperature algorithm 52 to determine or calculate an estimated Product core temperature at time T₁ based on one or both temperature values 504A and 506A, and/or measurements from additional sensor(s) 16.

Graph 500B represents the temperature situation at time T₂. As discussed above, Product 12 is exposed to an ambient temperature higher than the Product core temperature, such that temperature curve 502B is shifted upwards toward the ambient temperature, with respect to curve 502A. Temperature curve 502B extends from the 20° C. ambient temperature on both sides of Product 12, through the core of the Product, in this example a core temperature of 12.5° C. indicated at 510B. As shown, temperature values 504B and 506B measured by product-interfacing temperature sensor 18A and MCU internal on-chip temperature sensor 18D, respectively, have increased from values 504A and 506A shown in graph 500A. In the illustrated example, temperature sensor 18A measures a value 504B of approx. 10° C., and temperature sensor 18D measures a value 506B of approx. 14° C. PMD 10 may execute any suitable core temperature algorithm 52 to determine or calculate an estimated Product core temperature at time T₂ based on one or both temperature values 504B and 506B, and/or measurements from additional sensor(s) 16.

FIG. 23 is similar to FIG. 22, but illustrates a situation in which the Product is resting on a surface of solid structure 512 (e.g., shelf or table), as opposed to hanging in open air, according to an example embodiment. Thus, FIG. 23 illustrates an example temperature curve extending through a Product core, where the Product is thermally influenced by a combination of convection and conduction, i.e., by the transfer of heat (internal energy) by microscopic collisions of particles and movement of electrons within a body (the molecules of a solid surface transferring energy to the liquid inside the Product enclosure). FIG. 23 shows a first graph 500C showing a temperature curve 502C relative to the Product at a first time T₁, and a second graph 500C showing a temperature curve 502D relative to the Product at a second T₂ subsequent to first time T₁, which pair of graphs illustrates an upward shift in the temperature curve over time from T₁ to T₂, toward the ambient temperature. FIG. 23 may represent a situation in which a cooled Product is suddenly exposed to a higher ambient temperature (e.g., when the Product is removed from a cooler and placed on a table in an ambient room), wherein graph 500C indicates the temperature situation shortly after the cooled Product is exposed to the higher ambient temperature, and 500D indicates the temperature after more time exposed to the higher ambient temperature.

Graph 500C shows a cross-section of an example Product 12, e.g., blood product, including a Product packaging 12A (e.g., blood bag) that contains a temperature-sensitive fluid 12B (e.g., blood), and a PMD 10 secured to the Product packaging 12A. PMD 10 may include a product-interfacing temperature sensor 18A arranged to measure a surface temperature of the Product 12, a microcontroller 22B including an MCU internal on-chip temperature sensor 18D, and/or one or more other sensors 16. Temperature curve 502C extends from an ambient temperature on one side of Product 12 (in this example a 20° C. ambient temperature), through the core of the Product (in this example a core temperature of 5° C. indicated at 510C), and through the solid structure 512, which is at ambient (20° C.) when the Product 12 is placed on the structure 512. As Product 12 is exposed to the ambient temperature higher than the Product core temperature, the Product core temperature will increase over time, toward the ambient temperature.

Graph 500C also shows temperature values 504C and 506C along temperature curve 502C measured by product-interfacing temperature sensor 18A and MCU internal on-chip temperature sensor 18D, respectively. In the illustrated example, temperature sensor 18A measures a value 504C of approx. 6° C., and temperature sensor 18D measures a value 506A of approx. 10.5° C. PMD 10 may execute any suitable core temperature algorithm 52 to determine or calculate an estimated Product core temperature at time T₁ based on one or both temperature values 504C and 506C, and/or measurements from additional sensor(s) 16.

Graph 500D represents the temperature situation at time T₂. As discussed above, Product 12 is exposed to an ambient temperature higher than the Product core temperature, such that temperature curve 502D is shifted upwards toward the ambient temperature, with respect to curve 502C. Temperature curve 502D extends from the 20° C. ambient temperature on both sides of Product 12, through the core of the Product, in this example a core temperature of 12.5° C. indicated at 510D. As shown, temperature values 504D and 506D measured by product-interfacing temperature sensor 18A and MCU internal on-chip temperature sensor 18D, respectively, have increased from values 504A and 506A shown in graph 500A. In the illustrated example, temperature sensor 18A measures a value 504D of approx. 10° C., and temperature sensor 18D measures a value 506D of approx. 15° C. PMD 10 may execute any suitable core temperature algorithm 52 to determine or calculate an estimated Product core temperature at time T₂ based on one or both temperature values 504D and 506D, and/or measurements from additional sensor(s) 16.

Product and environment factors variations may be detected and approximated by an algorithm 52 based on known data from the sensor(s) 16 and/or derived system approximations.
Heat transfer contributions may be assumed based on the orientation of the product, for example hanging (convective heat transfer on all sides) vs lying (combination of convective heat transfer on one side and conductive heat transfer on the other side).
Orientation may be inferred from the inertial measurement unit (e.g. accelerometer etc., or tilt switch).
Heat transfer type and thermal dynamics may be inferred from the measured spatial gradients from different sides of the product as well as temperature dynamics.
Product mass/volume may be estimated/derived from the thermal dynamics and spatial gradients.

Some embodiments may use iterative methods in the core temperature derivation algorithm to derive or back-calculate certain variational parameters. For example, PMD 10 may assume certain mass of the product and/or type of heat transfer in the beginning but re-define them more accurately based on the output parameters of the algorithm (e.g. thermal dynamics and differences in measured vs calculated values). This approach may allow the derived parameters from model's output to be fed back as a re-defined input parameter until the calculations converge and no further adjustments are necessary.

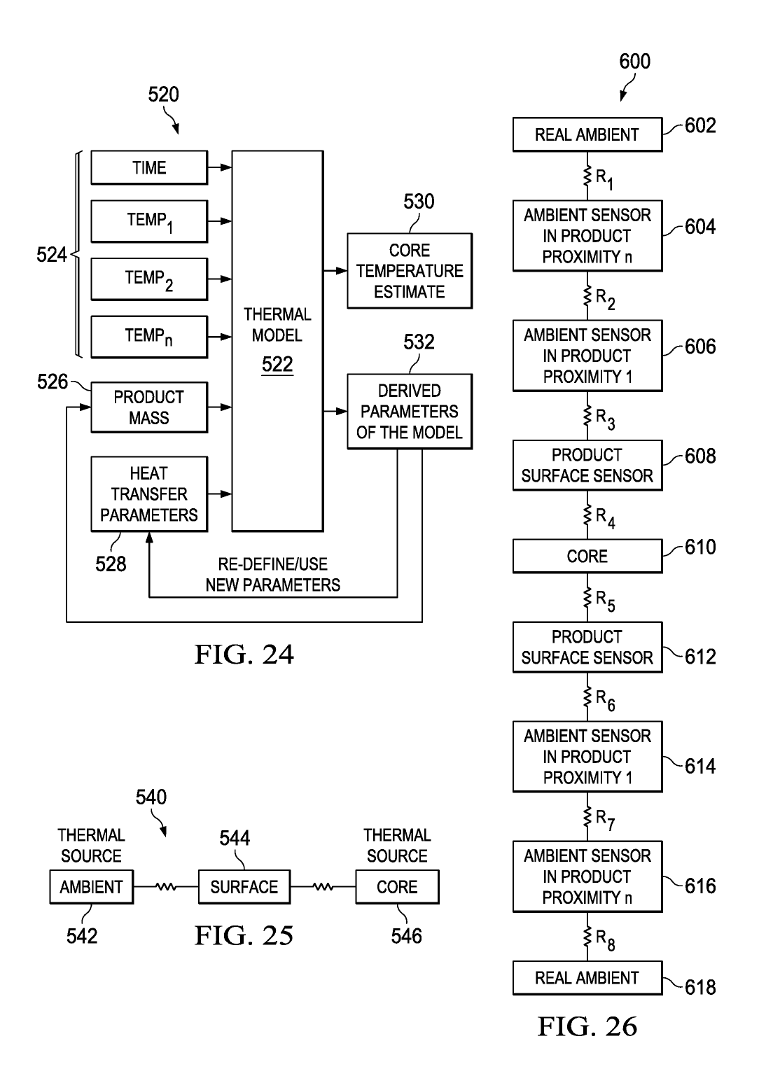

FIG. 24 illustrates an example algorithm 520 executable by a PMD processing unit 22 for calculating a core temperature of a Product using a thermal model, according to one embodiment. According to algorithm 520, a thermal model 522 may receive inputs 524, 526, and 528 and generate outputs 530 and 532. Inputs 524 may include temperature readings from any number of temperature sensors 18 (which generate Temp₁, Temp₂, . . . Tempₙ readings shown as inputs 524), along with a time input indicating current clock-based time data. The Product mass 526 (which may be automatically determined by PMD 10, assigned a predefined mass based on the type of Product, or input via a user interface 26) may also be fed into the thermal model 522, along with one or more heat transfer parameters 528, which may be dynamically adjusted based on the model outputs.

Thermal model 522 may receive inputs 524, 526, and 528, correlate the received temperature sensor readings with the received time data, and input the temperature sensor data, Product mass 526, and heat transfer parameters 528 into one or more heat equations (e.g., any of the heat equation(s) disclosed herein and/or any other heat equation(s) known to those of ordinary skill in the art) to calculate an estimated Product core temperature. As shown, algorithm 520 may implement a feedback loop by comparing thermal trends of the Product predicted with the thermal mode 522 with the actual thermal trends of the Product detected by the system. If PMD 10 determines a significant heating or cooling difference (e.g., exceeding some predefined threshold value(s)), PMD 10 may adjust thermal model 522 in real-time to better match the actual detected heating or cooling pattern of the Product. This may be particularly useful where the Product may be partially used and then put back into storage. In this case, algorithm 520 may dynamically adjust the Product mass and heat transfer parameters to match the current mass of the Product.

Generic Heating/Cooling Equation

Some embodiments of PMD 10 may execute an algorithm 52 based on one or more heat equation(s) to solve for the Product core temperature based on data from the PMD temperature sensor(s) 18 describing spatial and time gradients of the Product. For example, PMD 10 may execute a core temperature algorithm based on the classical Newton's cooling/heating equation:

$$\frac{\partial u}{\partial t} = α {∇}^{2} u = α ( \frac{{\partial }^{2} u}{\partial {x}^{2}} + \frac{{\partial }^{2} u}{\partial {y}^{2}} + \frac{{\partial }^{2} u}{\partial {z}^{2}} ) = α ( {u}_{xx} + {u}_{yy} + {u}_{zz} )$$

where:
u=u(x, y, z, t) is temperature as a function of space and time;

$$\frac{\partial u}{\partial t}$$

is the rate of change of temperature at a point over time;
uₓₓ, u_{yy}, and u_{zz} are the second spatial derivatives (thermal conductions) of temperature in the x, y, and z directions, respectively;

$$α = \frac{k}{{c}_{p} ρ}$$

is the thermal diffusivity, a material-specific quantity depending on the thermal conductivity k, the mass density ρ, and the specific heat capacity cₚ.

Newton's Heating/Cooling Model

The classical Newton's cooling/heating equation relies on the assumption that the gradient within the object is negligible so it

The heat-transfer version of Newton's law, which (as noted) requires a constant heat transfer coefficient, states that the rate of heat loss of a body is proportional to the difference in temperatures between the body and its surroundings.

The rate of heat transfer in such circumstances is derived below:

$$\frac{dQ}{dt} = h \cdot A \cdot ( T ( t ) - {T}_{env} ) = h \cdot A Δ T ( t )$$

Newton's cooling law in convection is a restatement of the differential equation given by Fourier's law:

$$\frac{dQ}{dt} = h \cdot A \cdot ( T ( t ) - {T}_{env} ) = h \cdot A Δ T ( t )$$

where
Q is the thermal energy in joules
h is the heat transfer coefficient (assumed independent of T here)(W/(m²K))
A is the heat transfer surface area (m²)
T is the temperature of the object's surface and interior (since these are the same in this approximation)
Tₑₙᵥ is the temperature of the environment; i.e. the temperature suitably far from the surface
ΔT(t)=T(t)−Tₑₙᵥ is the time-dependent thermal gradient between environment and object

The heat transfer coefficient h depends upon physical properties of the fluid and the physical situation in which convection occurs. Therefore, a single usable heat transfer coefficient (one that does not vary significantly across the temperature-difference ranges covered during cooling and heating) must be derived or found experimentally for every system that can be analyzed using the presumption that Newton's law will hold.

Static Resistive Lumped Model

The lumped capacitance model is a common approximation in transient conduction, which may be used whenever heat conduction within an object is much faster than heat transfer across the boundary of the object. The method of approximation then suitably reduces one aspect of the transient conduction system (spatial temperature variation within the object) to a more mathematically tractable form (that is, it is assumed that the temperature within the object is completely uniform in space, although this spatially uniform temperature value changes over time). The rising uniform temperature within the object or part of a system, can then be treated like a capacitative reservoir which absorbs heat until it reaches a steady thermal state in time (after which temperature does not change within it).

A useful concept used in heat transfer applications once the condition of steady state heat conduction has been reached, is the representation of thermal transfer by what is known as thermal circuits. A thermal circuit is the representation of the resistance to heat flow in each element of a circuit, as though it were an electrical resistor.

The lack of “capacitative” elements in the following purely resistive example, means that no section of the circuit is absorbing energy or changing in distribution of temperature. This is equivalent to demanding that a state of steady state heat conduction (or transfer, as in radiation) has already been established.

The equations describing the three heat transfer modes and their thermal resistances in steady state conditions, as discussed previously, are summarized in the table below:

Example equations for different heat transfer modes and their thermal resistances are set forth below in Table 1.

TABLE 1
Equations for different heat transfer modes and their thermal resistances.

| Transfer | Rate of Heat | |
|---|---|---|
| Mode | Transfer | Thermal Resistance |
| Conduc- tion | $\dot{Q} = \frac{{T}_{1} - {T}_{2}}{( \frac{L}{kA} )}$ | $\frac{L}{kA}$ |
| Convec- tion | $\dot{Q} = \frac{{T}_{surf} - {T}_{envr}}{( \frac{1}{{h}_{conv} {A}_{surf}} )}$ | $\frac{1}{{h}_{conv} {A}_{surf}}$ |
| Radia- tion | $\dot{Q} = \frac{{T}_{surf} - {T}_{surr}}{( \frac{1}{{h}_{r} {A}_{surf}} )}$ | $\frac{1}{{h}_{r} A} , where$ ${h}_{r} = εσ ( {T}_{surf}^{2} + {T}_{surr}^{2} ) ( {T}_{surf} + {T}_{surr} )$ |

FIG. 25 illustrates an example static thermal model 540 used to describe the thermodynamic system of an example Product. According to static thermal model 540, the ambient environment 542 is thermally coupled to the Product surface 544, which is in turn thermally coupled to the Product core 546, wherein each thermal coupling defines a resistance to heat transfer represented by an electrical resistor symbol.

According to model 540, an estimated core temperature can be calculated the as a value proportional to the difference between the measured ambient and surface temperatures. The proportionality factor reflects the mass and/or volume of the monitored product and the thermal environment conditions. The factor may be different for heating and cooling cycles.

The single capacitance approach can be expanded to involve many resistive and capacitive elements, with Bi<0.1 for each lump. As the Biot number is calculated based upon a characteristic length of the system, the system can often be broken into a sufficient number of sections, or lumps, so that the Biot number is acceptably small.

The resistive model may be further extended to capture physical system and processes more precisely. For example, the resistive model may be divided into more elements to determine spatial gradient profiles and types of heat transfer more precisely. Such model may satisfy certain physical approximation model assumptions and requirements and apply different models/parameters per each element.

FIG. 26 illustrates an example static thermal model 600 used to describe the thermodynamic system of an example Product. According to static thermal model 600, the ambient environment 602 on a first side of the Product is thermally coupled to one or more ambient sensors 604, 606 on the first side of the Product, in order of proximity to the Product, wherein the closest proximity ambient sensor 606 is thermally coupled to a product-interfacing sensor 608 on the first side of the Product, which is in turn thermally coupled to the Product core 610, which is thermally coupled to a product-interfacing sensor 612 on a second side of the Product, which is in turn thermally coupled to one or more ambient sensors 614, 616 on the second side of the Product, in order of proximity to the Product, with the furthest proximity ambient sensor 616 being thermally coupled to the ambient environment 618 on the second side of the Product.

In the model shown in FIG. 26, PMD 10 may use convection equivalent resistances for R1, R2, R3, and a conduction equivalent resistance for R4, i.e., a surface to core junction in the body of the Product. Similarly, the model may be symmetric from both sides with reference to the Product core, e.g., for a case in which the Product is suspended and surrounded by air from all sides. Alternatively, for a case in which the first side of the Product is exposed to ambient air convection, and the second side of the Product is lying on a surface, thermal resistances R1, R2, R3 associated with the first side of the Product may be determined by a thermal convection process, while the thermal resistances R6, R7, R8 associated with the second side of the Product in thermal contact with a surface may be determined by a thermal conduction process.

FIG. 27 illustrates an example algorithm 650 for monitoring the core temperate of a Product using a PMD 10 including at least one product-interfacing temperature sensor 18A configured to measure the Product surface temperature and at least one ambient temperature sensor 18C, according to an example embodiment. At 652, PMD 10 is powered on, e.g., in response to a user input or automatically upon detection of a triggering event, e.g., as disclosed herein. At 654, a processing unit 22 may read measurement values from temperature sensors 18A and 18C.

At 656, processing unit 22 may calculate an initial Product core temperature value T_{core} according to the following equation:

T_{core}=T_{surface}_{_}ᵢₙᵢₜᵢₐₗ+(T_{surface}_{_}ᵢₙᵢₜᵢₐₗ−T_{ambient}_{_}ᵢₙᵢₜᵢₐₗ)*C

where:
T_{surface}_{_}ᵢₙᵢₜᵢₐₗ is the Product surface temperature as measured by product-interfacing temperature sensor(s) 18A at 654 (in the case of multiple sensors 18A, T_{surface} may be an average or other mathematical combination of the multiple surface temperature measurement values);
T_{ambient}_{_}ᵢₙᵢₜᵢₐₗ is the ambient temperature of the ambient environment in which the Product is located, as measured by ambient temperature sensor(s) 18C at 654 (in the case of multiple ambient sensors 18C, T_{ambient} may be an average, weighted average, or other mathematical combination of the multiple ambient temperature measurement values)
C is a constant, which may be predefined as the heating constant C_{heating} or cooling constant C_{cooling} used in step 668 as discussed below, or any other suitable constant value.

At 658, processing unit 22 may determine an initial Product temperate status (e.g., safe temperature, existing temperature violation, imminent temperature violation, etc.) and control one or more LEDs or other output 28 to indicate the determined initial Product temperate status. At 660, PMD 10 may enter a sleep mode (e.g., low power mode) for a defined time period, e.g., to save battery power). At 662, PMD 10 may wake from the sleep mode based on a timer, and read temperature sensors 18A and 18C at 664.

At 666, processing unit 22 may identify and respond to any defined unexpected conditions, e.g., (a) a hand touch, (b) an out-of-range temperate reading (e.g., a thermistor reading of 300° C. or −50° C., (c) a noisy signal (e.g., temperature readings that vary by more than a defined amount over a defined time period), (d) exceedingly fast changes in surface temperature, e.g., as defined by rate of change threshold value(s), etc.

Algorithm 650 may be configured to filter out such data unexpected condition data, or handle such data in a way that does not jeopardize the Product. For example, hand-touch detection may be calibrated specifically to increase the weight of the core IIR filter such that the core estimate is predicted in a way correlating to slight heating from the hand but avoiding generation of a Product wastage alert off while the hand-touch persists. Once the unexpected condition subsides, algorithm 650 may return to normal operation. In the case of thermistors, if one or more thermistors is determined to be “bad” based on readings from such thermistor(s), the remaining temperature sensors may be used together to estimate the temperature as accurately as possible.

For at least some temperature monitoring situations, different formulas apply to a Product undergoing heating versus a Product undergoing cooling. For example, the following equations may be used to estimates the Product core temperature during heating and cooling, respectively, where the heating or cooling constants C_{heating} and C_{cooling} may be different:

T_{core}=T_{surface}+(T_{surface}−T_{ambient})*C_{heating}

T_{core}=T_{surface}+(T_{surface}−T_{ambient})*C_{cooling}

Thus, at 667, processing unit 22 may determine whether the Product is heating or cooling, e.g., by comparing surface temperature sensor 18A readings taken at 664 with previous surface temperature sensor 18A (readings e.g., readings taken at 664 or a previous instance of step 664), and select a constant C_{heating} or cooling constant C_{cooling} (for use in subsequent step 668) based on the heating/cooling determination. In one embodiment, algorithm 650 defines a heating constant C_{heating} and a cooling constant C_{cooling}, and determines which of these two constants to apply at step 668 based on the heating/cooling determination at 667.

At 668, processing unit 22 may calculate a new Product core temperature value T_{core} according to the following equation:

T_{core}_{_}_{new}=T_{surface}_{_}_{current}+(T_{surface}_{_}_{current}−T_{ambient}_{_}_{current})*C_{heating}/C_{cooling}

where:
T_{surface}_{_}_{current} is the current Product surface temperature as measured by product-interfacing temperature sensor(s) 18A at 664 (in the case of multiple sensors 18A, T_{surface} may be an average or other mathematical combination of the multiple surface temperature measurement values);
T_{ambient}_{_}_{current} is the ambient temperature of the environment in which the Product is located, as measured by ambient temperature sensor(s) 18C at 664 (in the case of multiple ambient sensors 18C, T_{ambient} may be an average, weighted average, or other mathematical combination of the multiple ambient temperature measurement values);
C_{heating}/C_{cooling} represents the heating or cooling constant selected at 667.

At 670, processing unit 22 may execute an IIR filter according to the equation:

T_{core}_{_}_{filtered}=T_{core}−(T_{core}−T_{core}_{_}_{new})*(Filter Weight)

where “Filter Weight” is a weight that may be set to mimic the average mass of the type of Product being monitored (e.g., the average mass of a blood bag) and suppress high frequency transients. In some embodiments, processing unit 22 may dynamically adjust the Filter Weight based on determined heating or cooling patterns of the Product, as detected by PMD 10.

At 672, processing unit 22 may check for an existing or imminent Product wastage condition, e.g., according to any of the techniques or algorithms disclosed herein, and generate and output a corresponding notification or alarm if an existing or imminent Product wastage condition is determined. For example, an existing Product wastage condition may be determined if the T_{core}_{_}_{filtered} determined at 670 exceeds a predefined Product wastage threshold value. An imminent Product wastage condition may be determined by calculating a trend of T_{core}_{_}_{filtered} (e.g., based on the current T_{core}_{_}_{filtered} value and one or more previously calculated T_{core} _{_}_{filtered} values); based on this calculated trend, calculating a time until the Product temperature is expected to exceed the predefined Product wastage threshold value, and comparing this time to an imminent Product wastage threshold time value (e.g., 15 minutes).

In some embodiments, e.g., embodiments in which algorithm 650 is executed by an 8-bit microcontroller, constants C_{heating} and C_{cooling} may be defined by 2s complement and can be mathematically represented as 1/2ⁿ to allow calculations through simple bit shifting operations. If more complex constants are used, e.g., to improve accuracy, the value of such constant may be effected by execution of a series summation of 1/2ⁿ values, with any combination of values for n to provide the desired constant (e.g., between 0 and 1).

Thus, in such embodiments, the term (T_{surface}−T_{ambient})*C_{heating}/C_{cooling} as applied in step 656 or 668 may be written as:

$$Σ ( ( {C}_{SURFACE} - {C}_{AMBIENT} ) * \frac{1}{{2}^{n}} )$$

where the summation may include any number of iterations corresponding to any defined set of values for n. For example, to implement a heating constant of 0.375, which is equivalent to 0.25+0.125 in terms of 1/(2ⁿ), it can be represented by 1/(2²)+1/(2³), such that the summation above includes two iterations: a first iteration with n=2 and a second iteration with n=3.

Example Prototype

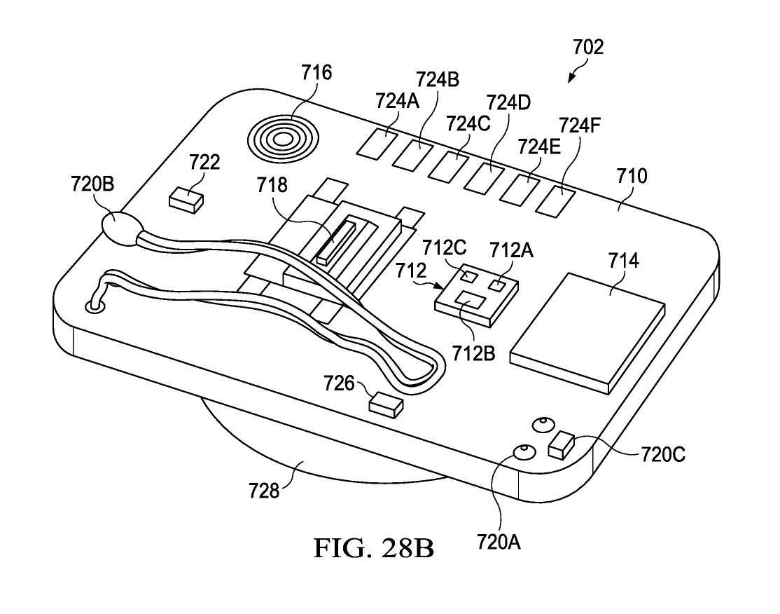

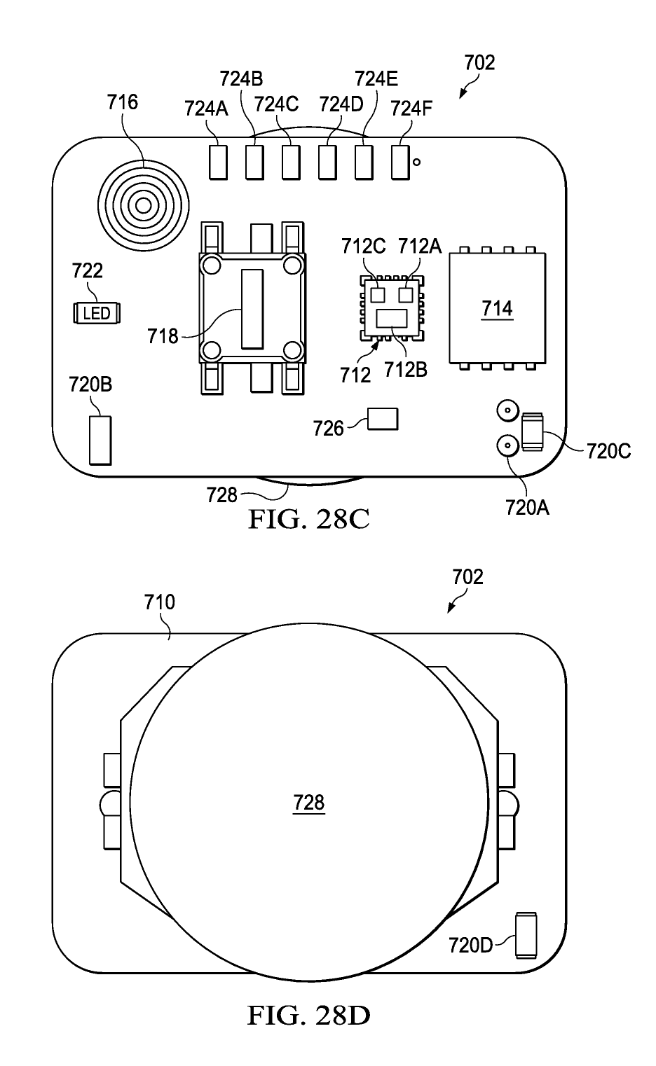

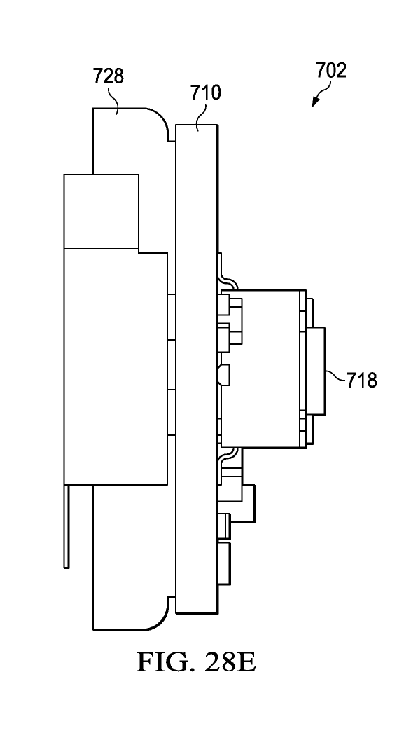

An example prototype of a PMD 700 illustrated and discussed below with respect to FIGS. 28A-28E. FIG. 28A shows a three-dimensional view of PMD 700 from above, which includes a PMD assembly 702 housed in a PMD enclosure 704; FIG. 28B shows a three-dimensional view of PMD assembly 702, with PMD packing 704 removed for the purposes of illustration; and FIGS. 28CA-28E illustrate top, bottom, and side views of PMD assembly 702.

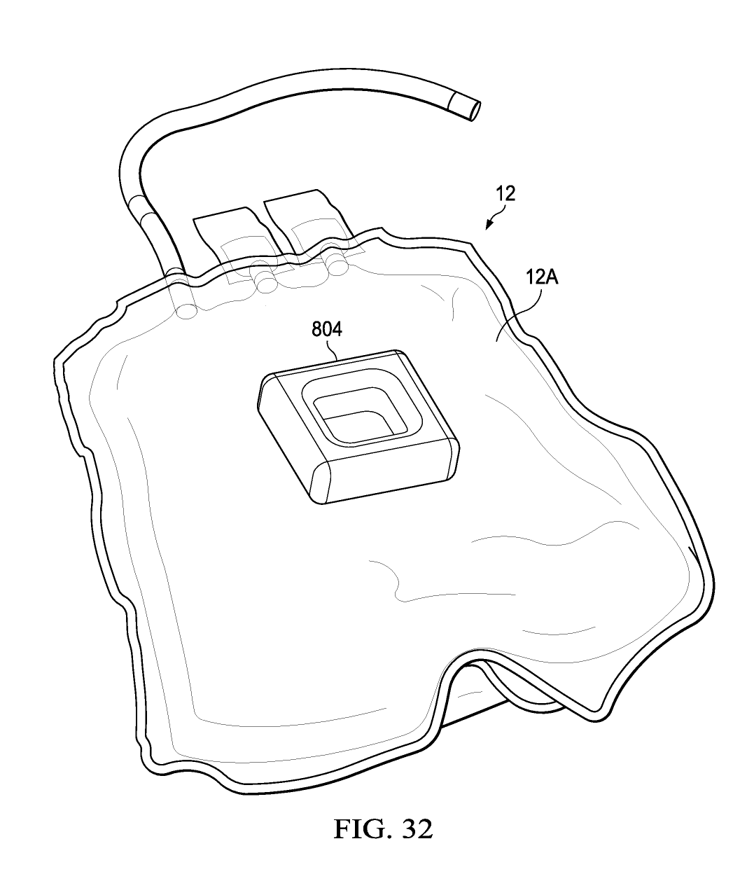

As shown in FIG. 28A, PMD 700 may include a PMD assembly 702 housed in a PMD enclosure 704, either permanently or removably. PMD enclosure 704 may be configured for attaching PMD 700 to a Product packaging (e.g., blood bag) for monitoring an Product (e.g., blood). In the illustrated example, PMD enclosure 704 includes an attachment portion 706 configured for attachment to the Product packaging or to an attachment structure configured to attach to the Product packaging. The Product packaging may including a PMD-locating feature for physically locating PMD 700 relative to the Product in a defined location and/or orientation. An example of such PMD-locating feature is shown in FIG. 32, discussed below.

In one embodiment, a bottom surface of attachment portion 706 may have an adhesive surface, or configured to receive an adhesive applied thereto, for direct or indirect attachment to the Product packaging. For example, a thin (<5 mil) double sided adhesive tape may be applied to the bottom surface of attachment portion 706 and to a surface of the Product packaging, which may provide a thermal contact between one or more sensors 16 (e.g., a thermistor) and the Product.

PMD enclosure 704 may also be configured to protect PMD assembly 702 from the environment. For example, PMD enclosure 704 may provide a water-tight and/or air-tight sealed enclosure for PMD assembly 702. In some embodiments, PMD enclosure 704 is disposable such that PMD assembly 702 may be removed from a first PMD enclosure 704 and arranged in a second PMD enclosure 704, e.g., to dispose of the first PMD enclosure 704 after a period of use of PMD 700.

The top surface contains a button that permits interrogating the state of the monitored blood product. LED indicator(s) (e.g., LED1) may communicate the state of the blood product.

As shown in FIGS. 28B and 28C, PMD assembly 702 may include various electrical components integrated in, arranged on, or otherwise secured to a circuit board 710, card, or other structure. For example, PMD assembly 702 may include any or all of the following components: a microcontroller 712, a flash memory device 714, a capacitive sensor 716, a manually pressable button 718, one or more thermistors 720 (distinct from MCU 712), at least one LED 722, a plurality of interface contacts 724, a wireless interface 726, a battery 728, and/or any other electronics for providing any of the functionality disclosed herein or any related functionality, as would be understood by one of ordinary skill in the art.

In one embodiment, capacitive sensor 716 is a touch pad that functions as a user input device, e.g., for activating PMD 700 (e.g., by waking PMD 700 from a sleep or deactivated state). In such embodiment, capacitive sensor 716 and push button 718 may both function as user input device, e.g., to activate PMD 700. In another embodiment, capacitive sensor 716 is configured to detect the presence of a Product, e.g., for automatic activation of PMD 700.

Microcontroller (MCU) 712 may include a CPU 712A for calculating Product temperature values and/or performing other functions of PMD 700, a memory device 712B, an internal on-chip temperature sensor 712C, input/output elements, and/or any other MCU electronics.

CPU 712A (or another processing unit 22 of PMD 700, e.g., a microprocessor separate from MCU 712) may be configured to execute algorithms or other computer instructions to calculate at least one temperature characteristic of a respective Product and/or the environment in which the Product is located. For example, CPU 712A may calculate or determine one or more of (a) a current Product surface temperature, (b) a current Product core temperature, (c) a predicted future Product surface temperature, (d) a predicted future Product core temperature, and/or (e) an ambient environment temperature, wherein each of such temperature characteristics may be calculated as a function of one or more other temperature characteristics (e.g., CPU 712A may measure or calculate an ambient environment temperature, and then calculate a current or predicted future Product core temperature based at least on the determined ambient environment temperature.

In some embodiments, CPU 712A (or other processing unit 22) may use sensor data from MCU internal on-chip temperature sensor 712C, either by itself or in combination with sensor data from additional temperature sensor(s) (e.g., one or more thermistors 720) and/or other sensor(s)), for calculating any of the various temperature characteristics discussed above, e.g., using any of the temperature calculation algorithms disclosed herein.

Flash memory 714 and/or MCU on-chip memory 712B may store any data relevant to PMD 700, e.g., any of the types of data 50-66 discussed above with reference to FIG. 3.

LED(s) 722 may act as output device(s) to communicate one or more types of information, e.g., to indicate the operational state of the PMD (e.g., sleep mode vs. activated mode), to indicate a current temperature violation or Product wastage condition, and/or to warn of a predicted further temperature violation or Product wastage condition.

Wireless interface 726 may include antenna(s) and related electronics for wirelessly transmitting data from PMD 700 (e.g., determined temperatures and/or alert notifications) and/or wirelessly receiving data at PMD 700 (e.g., control data for controlling the functionality of PMD 700.

Interface contacts 724 may include contact for physically interfacing with PMD 700, e.g., by external devices. In this example, interface contacts 724 include:
a data interface 724A and clock interface 724B for connection to a chip programmer/debugger, e.g., for programming and debugging PMD 700 from an external host interface, and/or to provide direct access the internal MCU memory 712B, e.g., to read the contents of memory 712B;
a transmit (TX) line contact 724C for sending data from MCU 712 to an external interface, e.g., via the “universal asynchronous receiver/transmitter” (UART) communication protocol;
a receive (RX) line contact 724D for sending data from an external interface to MCU 712, e.g., via the UART communication protocol;
a signal ground line (GND) contact 724E; and
a PMD power line (VCC) contact 724F.

VCC and GND lines are both directly connected to the battery terminals of PMD batter 728. With the battery 728 inserted, PMD 700 may use VCC to sense the battery voltage. Without the battery 728 inserted, VCC can be used to power the PMD externally via VCC contact 724F. In the illustrated example, PMD battery 728 is located on the back side of the board 710; in other embodiments battery 728 may be arranged in any other suitable location or orientation.

PMD 70 may include one or more thermistors 720, which may be used in combination with MCU internal on-chip temperature sensor 712B for determining one or more relevant temperature characteristics, as discussed above. In one embodiment, PMD 70 includes at least a Product-surface sensor 720A and an ambient temperature sensor 720B, and/or one or more additional thermistors 720C and/or 720D.

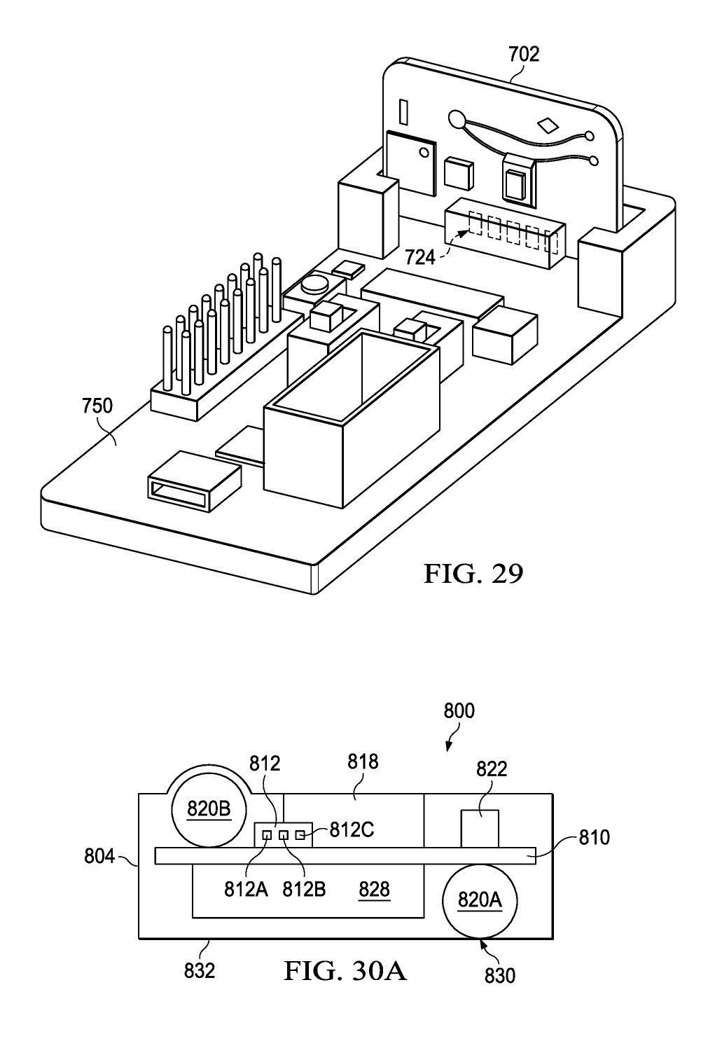

FIG. 29 illustrates an example debug/programming device 750 configured to receive PMD assembly 702 (removed from PMD enclosure 704) with a plug-in type connection, which creates a physical contact between one or more interface contacts 724 of PMD assembly 702 with corresponding contacts provided by debug/programming device 750, to thereby define physically interfaces for communicating data, signals, and/or power to and/or from PMD assembly 702.

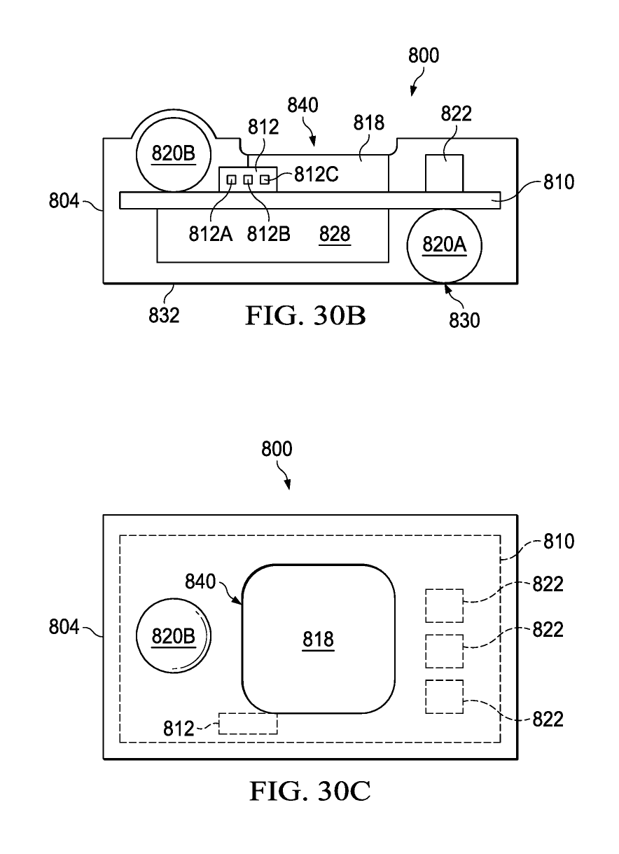

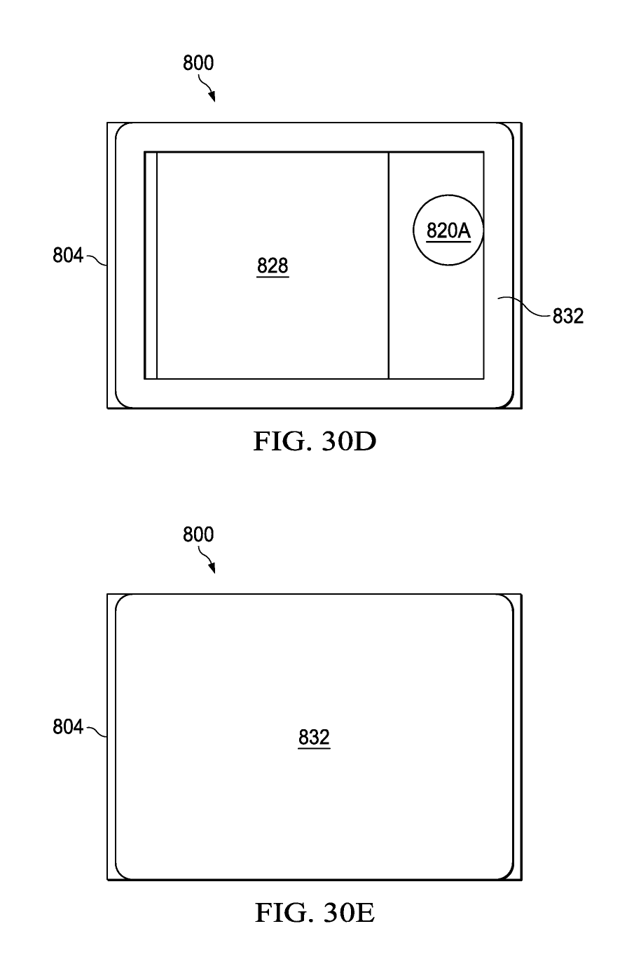

FIGS. 30A-30E illustrate various views of another example PMD 800 for monitoring the temperature of a Product, e.g., a blood product, according to example embodiments. Referring first to FIG. 30A, which shows a cross-sectional side view of PMD 800 according to one embodiment, PMD 800 may include a PMD assembly 802 arranged in an enclosure 804, e.g., a sealed enclosure. PMD assembly 802 may include various electronics integrated in and/or secured to a printed circuit board (PCB) 810, including a microcontroller 812, a push button 818, a Product-interfacing thermistor 820A, an ambient temperature thermistor 820B, one or more LED indicators 822, and/or any other electronics for providing any of the functionality disclosed herein or any related functionality, as would be understood by one of ordinary skill in the art.

Microcontroller (MCU) 812 may include a CPU 812A for calculating Product temperature values and/or performing other functions of PMD 800, a memory device 812B, an internal on-chip temperature sensor 812C, input/output elements, and/or any other MCU electronics. CPU 812A may calculate or determine one or more of (a) a current Product surface temperature, (b) a current Product core temperature, (c) a predicted future Product surface temperature, (d) a predicted future Product core temperature, and/or (e) an ambient environment temperature, wherein each of such temperature characteristics may be calculated as a function of one or more other temperature characteristics (e.g., CPU 812A may measure or calculate an ambient environment temperature, and then calculate a current or predicted future Product core temperature based at least on the determined ambient environment temperature.)

Product-interfacing thermistor 820A may be arranged to measure a surface temperature of the Product. For example, product-interfacing thermistor 820A may be attached to a bottom side or otherwise located below PCB 810, to thereby interface with the Product surface at point 830 shown in FIG. 30A. Ambient temperature thermistor 820B may be arranged to measure an ambient temperature of the environment in which the Product and PMD 800 are located. Thus, ambient temperature thermistor 820B may be attached to a top side or otherwise located above PCB 810 (opposite the bottom side that faces the Product).

In some embodiments, CPU 812A (or other processing unit 22) may use sensor data from one, two, or all three of MCU internal on-chip temperature sensor 812C, Product-interfacing thermistor 820A, and ambient temperature thermistor 820B for calculating any of the various temperature characteristics discussed above, e.g., using any of the temperature calculation algorithms disclosed herein.

MCU on-chip memory 812B may store any data relevant to PMD 800, e.g., any of the types of data 50-66 discussed above with reference to FIG. 3.

Push button 818 may function as a user input device, e.g., to activate and/or deactivate PMD 800. As shown in FIG. 30A, push button 818 may be located below a flexible portion of the PMD enclosure 804 (e.g., a thin plastic or rubber film), such that the PMD enclosure 804 may flex upon when user presses button 818.

LEDs 822 may act as output devices to communicate one or more types of information, e.g., to indicate the operational state of the PMD (e.g., sleep mode vs. activated mode), to indicate a current temperature violation or Product wastage condition, and/or to warn of a predicted further temperature violation or Product wastage condition.

PMD 800 may be attachment to the Product packaging or to an attachment structure configured to attach to the Product packaging in any suitable manner. For example, two-sided tape or other adhesive, indicated at 832, may be arranged on a bottom surface of PMD enclosure 804 to secure PMD enclosure 804 directly to the Product or to a suitable PMD attachment structure attached to the Product.

FIG. 30B illustrates an alternative embodiment, which may be similar to the embodiment of FIG. 30A except for including a recess or indentation 840 in the PMD enclosure 804 above the push button 818, e.g., to provide visual and tactile guidance for locating button 818 and/or to protect button 818 from accidental actuation.

FIG. 30C shows a top view of PMD 800, showing various components arranged on or above a top side of PCB 810, according to one embodiment.

FIG. 30D shows a bottom view of PMD 800, showing various components arranged on or below a bottom side of PCB 810, according to one embodiment. In this embodiment, a die-cut ring of adhesive 832 is attached to a bottom surface of PMD enclosure 804 and extends around a perimeter of this bottom surface.

FIG. 30E shows a top view of PMD 800, showing various components arranged on or below a bottom side of PCB 810, according to another embodiment. In this embodiment, an adhesive film 832 covers the full bottom surface of PMD enclosure 804.

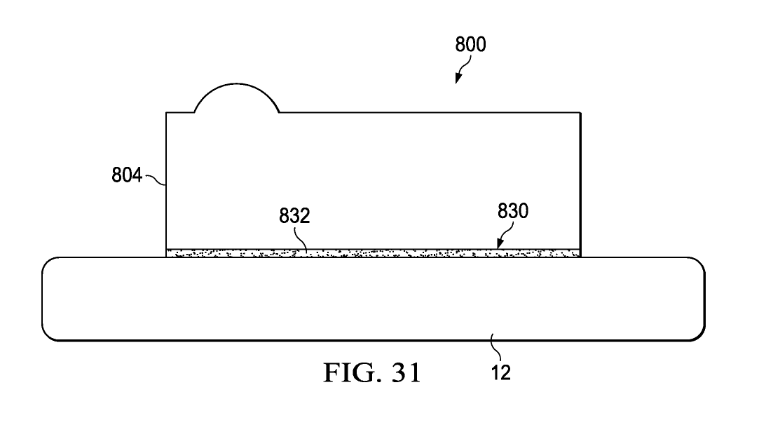

FIG. 31 illustrates an external side view of PMD 800 attached to a blood pack 12, according to one embodiment. PMD 800 may be secured to blood pack 12 may any suitable type of adhesive 832. Reference number 830 represents a point of interface between a Product-interfacing temperature sensor 820A and the surface of blood pack 12, e.g., as discussed above with respect to the FIG. 30A.

FIG. 32 illustrates an example blood pack 12 (filled with saline) with the PMD enclosure 824 of an example PMD 800 attached to an outer surface 12A of the blood pack 12, e.g., via an adhesive 832 as discussed above. The internal components of PMD 804 arranged inside PMD enclosure 824 are removed for the purposes of this illustration.

In some embodiments or configurations, PMD 10 may be attached to an outer surface of a Product (e.g., blood pack) or may be suspended inside the Product in any other suitable manner. In some embodiments, PMD 10 may either be mounted on the outside surface of the Product packaging or may be directly built-in or embedded into the Product packaging. In either of the such configurations, one or more sensors 16 (e.g., one or more temperature sensors 18) of the PMD may be located within the Product packaging and directly inside the Product internal volume or core.

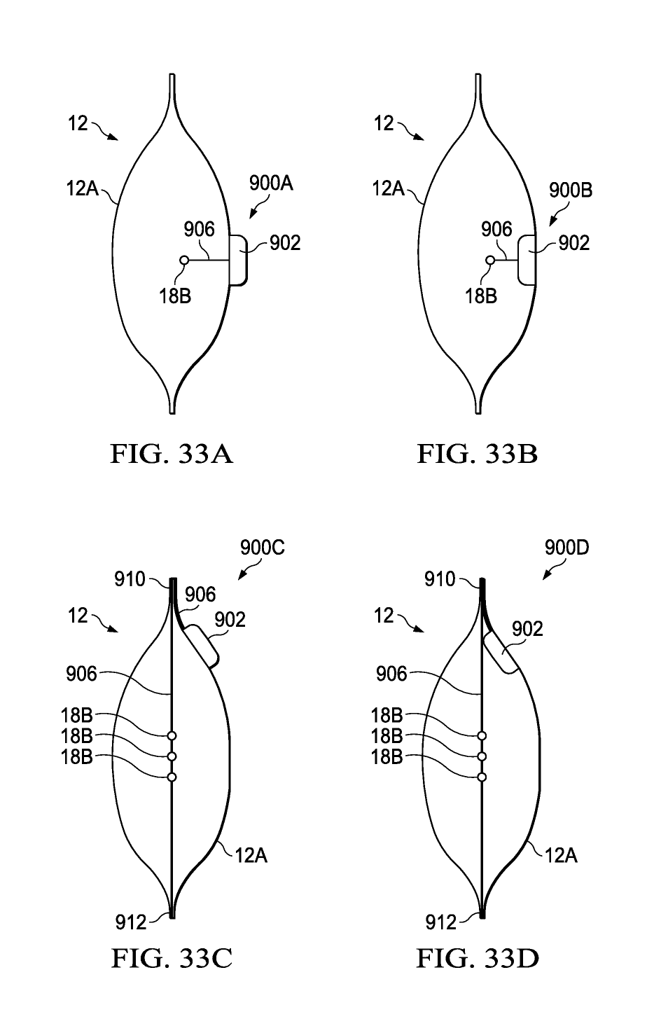

FIGS. 33A-33D illustrate example PMDs 900A-900D that include one or more internal Product temperature sensors 18B (shown also in FIG. 2) located inside the Product itself, e.g., inside a blood bag of a blood product, according to example embodiments. Internal Product temperature sensors 18B may be configured to directly measure an internal temperature of the respective Product, e.g., the Product core temperature.

FIG. 33A shows an example PMD 900A including a PMD body 902 arranged on an outer surface of the Product packaging 12A, e.g., the outer surface of a blood bag, and a single internal Product temperature sensor 18B connected to PMD body 902 by a conductor 906. PMD body 902 may include any of the various components and provide any of the various functionality of any embodiments of PMD 10 discussed herein. Conductor 906 may include one or more electrical conductors for communicating signals and/or power between PMD body 902 and internal Product temperature sensor 18B, which conductor(s) may be insulated or protected within a tube, sleeve, or protective coating, for example. Conductor 906 may be rigid or semi-rigid, e.g., to maintain sensor 18B in a particular location within the Product, e.g., at or near the geometric center of the Product. In some embodiments, PMD 900A may include multiple internal Product temperature sensors 18B connected to PMD body 902 by one or more conductors 906.

FIG. 33B shows an example PMD 900B, which may be similar to PMD 900A shown in FIG. 33A, but wherein PMD body 902 is arranged on an inner surface of the Product packaging 12A, e.g., the inner surface of a blood bag. PMD 900B may be fully enclosed within the Product packaging 12A. In the illustrated embodiment, PMD 900B includes a single internal Product temperature sensor 18B connected to PMD body 902 by a conductor 906. In an alternative embodiment, PMD 900B may include multiple internal Product temperature sensors 18B connected to PMD body 902 by one or more conductors 906.

FIG. 33C shows an example PMD 900C including a PMD body 902 arranged on an outer surface of the Product packaging 12A, e.g., the outer surface of a blood bag, and a multiple internal Product temperature sensor 18B arranged along a conductor 906 that is connected to PMD body 902. PMD body 902 may include any of the various components and provide any of the various functionality of any embodiments of PMD 10 discussed herein. Conductor 906 may include one or more electrical conductors for communicating signals and/or power between PMD body 902 and internal Product temperature sensors 18B, which conductor(s) may be insulated or protected within a tube, sleeve, or protective coating, for example. Conductor 906 may be connected at opposite ends of the Product packaging 12A, e.g., such that the internal Product temperature sensors 18B remain suspended in a desired location within the Product, e.g., proximate the geometric center of the three-dimensional volume of the Product. As shown, a portion of conductor 906 (e.g., a portion near PMD body 902) may run on the outer surface the Product packaging 12A. In another embodiment, rather than providing the structural support itself for the internal Product temperature sensors 18B, conductor 906 may be secured to a separate support structure within the Product, e.g., a line or internal wall that extends through the interior of the Product. In an alternative embodiment, PMD 900C may include a single internal Product temperature sensor 18B connected to PMD body 902 via conductor 906.

FIG. 33D shows an example PMD 900D, which may be similar to PMD 900C shown in FIG. 33C, but wherein PMD body 902 is arranged on an inner surface of the Product packaging 12A, e.g., the inner surface of a blood bag. In in this embodiment, PMD 900D may be fully enclosed within the Product packaging 12A, including the entire length of conductor 906.

In some embodiments, PMD 10 may be inserted or snapped into a mount or a fixture, e.g., to allow PMD 10 to be attached to and detached from a Product. Thus, PMD 10 may be reusable multiple times on the same instance of a Product or on multiple instances of a Product.

In addition, the Product packaging may be specifically designed to improve thermal conductivity to one or more temperature sensors 18, while maintaining water resistance of PMD 10. In some embodiment, this is achieved using molded grooves in the Product enclosure that isolate one or more temperature sensors in small recesses, pockets, or “chimneys” to increase the exposure of the surface area of the package/sensor to the ambient air. This type of structure may also be useful for aligning one or more temperature sensors within the Product package and keeping such sensor(s) in place throughout the lifecycle of PMD 10 by adjusting the tolerances to securely fit the sensor(s).

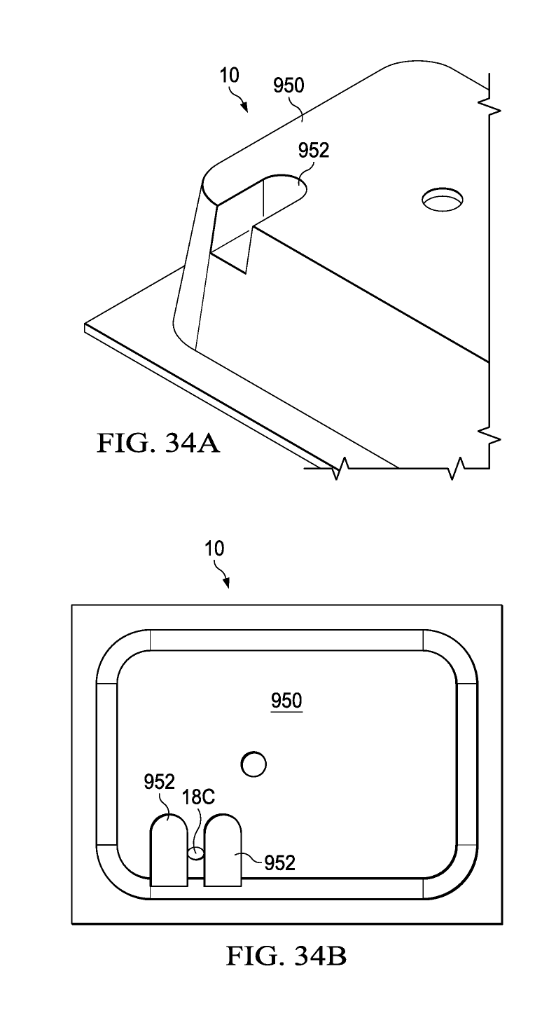

FIGS. 34A and 34B illustrate example embodiments of a PMD packaging 950 including one or more recesses 952 for providing increased exposure of one or more temperature sensors to the ambient environment. For example, FIG. 34A shows an example with a single recess 952 formed in the PMD packaging 950, while FIG. 34B shows an example with a pair of recesses 952 formed in the PMD packaging 950 to provide increased exposure of an ambient temperature sensor 18C to ambient air.

### Additional Example Applications

As discussed above, in some embodiments, PMDs 10 may be used to track the Product Characteristic(s) of shipment packages containing Product(s).

A tracking system may be configured to allow users to track the Product Characteristic(s) of Products via cloud-connected PMDs 10. The system may include databases(s) in which unique info for each PMD 10 is logged, and applications programmed to read such information and alert a user of status changes in the respective Products.

Further, in some embodiment, Product location may be tracked using GPS and other location methods such as iBeacons, for example.

1. A device for monitoring a temperature of a product, the device comprising:
at least one product-interfacing temperature sensor in thermal contact with the product and configured to generate first temperature sensor signals indicating a temperature associated with the product;
at least one ambient temperature sensor configured to generate second temperature sensor signals indicating a temperature of an ambient environment in which the product is located;
a processor; and
computer instructions stored in non-volatile computer-readable media and executable by the processor to:
receive the first sensor signals generated by the at least one product-interfacing temperature sensor;
receive the second sensor signals generated by the at least one ambient temperature sensor;
determine a product temperature of the product based at least on (a) the first sensor signals generated by the at least one product-interfacing temperature sensor and (b) the second sensor signals generated by the at least one ambient temperature sensor;
identify the existence of a notification condition based at least one the determined product temperature; and
generate a notification in response to identifying the existence of a notification condition.

2. The device of claim 1, wherein the product comprises a liquid or gas contained in a product enclosure.

3. The device of claim 1, wherein the product comprises a blood pack or other blood product.

4. The device of claim 1, wherein the determined product temperature comprises an estimated core temperature value of the product.

5. The device of claim 1, wherein identifying the existence of a notification condition based at least one the determined product temperature comprises comparing the determined product temperature to at least one threshold value.

6. The device of claim 1, wherein identifying the existence of a notification condition based at least one the determined product temperature comprises calculating a predicted product temperature associated with a defined future time.

7. The device of claim 1, wherein identifying the existence of a notification condition based at least one the determined product temperature comprises:
calculating a time-based trend in the determined product temperature; and
calculating a time until the time-based trend in the determined product temperature exceeds a threshold temperature value.

8. The device of claim 7, wherein identifying the existence of a notification condition based at least one the determined product temperature further comprises:
comparing the calculated time until the time-based trend in the determined product temperature exceeds the threshold temperature value to a time threshold value; and
generating a notification based on a result of the comparison of the calculated time until the time-based trend in the determined product temperature exceeds the threshold temperature value to the time threshold value.

9. The device of claim 1, wherein identifying the existence of a notification condition based at least one the determined product temperature comprises:
calculating a rate of change in the determined product temperature;
comparing the rate of change in the determined product temperature to a threshold rate of change; and
generating a notification in response to determining that the rate of change in the determined product temperature exceeds the threshold rate of change.

10. The device of claim 1, wherein the at least one product-interfacing temperature sensor comprises at least one temperature sensor configured to measure a surface temperature of the product.

11. The device of claim 1, wherein the at least one product-interfacing temperature sensor comprises at least one temperature sensor arranged within an enclosure of the product or within an interior volume of the product.

12. The device of claim 1, further comprising:
at least one orientation sensor configured to generate orientation sensor signals indicating a physical orientation of the product;
wherein the computer instructions are executable to determine the product temperature of the product based at least on (a) the first sensor signals generated by the at least one product-interfacing temperature sensor, (b) the second sensor signals generated by the at least one ambient temperature sensor, and (c) orientation sensor signals generated by the at least one orientation sensor.

13. The device of claim 1, wherein:
the computer instructions are executable to execute a temperature calculation algorithm to determine the product temperature of the product based at least on (a) the first sensor signals generated by the at least one product-interfacing temperature sensor, (b) the second sensor signals generated by the at least one ambient temperature sensor;
the device further includes at least one orientation sensor configured to generate orientation sensor signals indicating a physical orientation of the product; and
the computer instructions are further executable to automatically adjust the temperature calculation algorithm based on the orientation sensor signals generated by the at least one orientation sensor.

14. The device of claim 1, wherein the computer instructions are executable to determine the product temperature of the product based at least on (a) the first sensor signals generated by the at least one product-interfacing temperature sensor, (b) the second sensor signals generated by the at least one ambient temperature sensor, and (c) a mass or volume of the product.

15. A device for monitoring a temperature of a product, the device comprising:
a microcontroller (MCU) comprising an MCU on-chip temperature sensor configured to generate first sensor signals;
a product-interfacing temperature sensor in thermal contact with the product and configured to generate second sensor signals;
computer instructions stored in non-volatile computer-readable media and executable by a processor to:
receive the first sensor signals generated by the MCU on-chip temperature sensor;
receive the second sensor signals generated by the product-interfacing temperature sensor;
determine a product temperature of the product based on at least one of (a) the first sensor signals generated by the MCU on-chip temperature sensor or (b) the second sensor signals generated by the product-interfacing temperature sensor;
identify the existence of a notification condition based at least one the determined product temperature; and
generate a notification in response to identifying the existence of a notification condition.

16. The device of claim 15, wherein identifying the existence of a notification condition based at least one the determined product temperature comprises comparing the determined product temperature to at least one threshold value.

17. The device of claim 15, wherein identifying the existence of a notification condition based at least one the determined product temperature comprises:
calculating a time-based trend in the determined product temperature; and
calculating a time until the time-based trend in the determined product temperature exceeds a threshold temperature value.

18. The device of claim 17, wherein identifying the existence of a notification condition based at least one the determined product temperature further comprises:
comparing the calculated time until the time-based trend in the determined product temperature exceeds the threshold temperature value to a time threshold value; and
generating a notification based on a result of the comparison of the calculated time until the time-based trend in the determined product temperature exceeds the threshold temperature value to the time threshold value.

19. The device of claim 15, wherein the processor is provided on the MCU.

20. The device of claim 15, wherein the processor is separate from the MCU.

21. The device of claim 15, further comprising an ambient temperature sensor configured to generate third sensor signals indicative of an external environment of the product.

22. The device of claim 15, further comprising:
at least one orientation sensor configured to generate orientation sensor signals indicating a physical orientation of the product;
wherein the computer instructions are executable to determine the product temperature of the product based at least on (a) at least one of the first sensor signals generated by the MCU on-chip temperature sensor or the second sensor signals generated by the product-interfacing temperature sensor, and (b) orientation sensor signals generated by the at least one orientation sensor.

23. A method for monitoring a temperature of a product, the method comprising:
receiving, at a processing unit, the first sensor signals generated by the product temperature sensor;
receiving, at the processing unit, the second sensor signals generated by the ambient temperature sensor;
determining, by the processing unit, a predicted product temperature of the product based at least on (a) the first sensor signals and (b) the second sensor signals, the predicted product temperature indicating a predicted temperature of the product at a future time;
comparing, by the processing unit, the predicted product temperature with one or more threshold values;
determining, by the processing unit, based on the comparison, whether to generate an alert signal.

24. A device for monitoring a temperature of a product, the device comprising:
at least one temperature sensor configured to generate temperature sensor signals associated with the product;
a processor; and
computer instructions stored in non-volatile computer-readable media and executable by the processor to:
receive the temperature sensor signals generated by the at least one temperature sensor;
determine a product temperature of the product based at least on the temperature sensor signals;
calculate a time-based trend in the product temperature;
calculate, based at least on the time-based trend in the product temperature, a predicted time at which the product temperature will exceed a threshold temperature value; and
generating a notification based on the predicted time at which the product temperature will exceed a threshold temperature value.

25. The device of claim 24, wherein generating a notification based on the predicted time at which the product temperature will exceed a threshold temperature value comprises:
comparing a time until the predicted time to a time threshold value; and
generating a warning notification in response to determining that the time until the predicted time is less than the time threshold value.
